(19) United States
(12) Patent Application Publication
(10) Pub. No.: US 2022/0175818 A1
(43) Pub. Date: Jun. 9, 2022

(54) Inhibition of Monoamine Oxidase Subtype A (MAOA) Mitigates Cardiovascular Calcification

(71) Applicant: The Brigham and Women's Hospital, Inc., Boston, MA (US)

(72) Inventors: Julius Decano, Boston, MA (US); Elena Aikawa, Chestnut Hill, MA (US); Masanori Aikawa, Chestnut Hill, MA (US); Yukio Francisco Iwamoto, Meerbusch (DE)

(21) Appl. No.: 17/598,150

(22) PCT Filed: Mar. 27, 2020
(86) PCT No.: PCT/US2020/025295

§ 371 Date: Sep. 24, 2021

Related U.S. Application Data
(60) Provisional application No. 62/824,383, filed on Mar. 27, 2019.

(51) Int. Cl. A61K 31/7105; A61K 31/138; A61K 31/4985; A61K 31/5375; A61K 31/421; A61P 9/10

(52) U.S. Cl. CPC A61K 31/7105; A61K 31/138; A61P 9/10; A61K 31/5375; A61K 31/421; A61K 31/4985

(57) ABSTRACT

Compositions and methods for use in the treatment of disorders associated with aortic valve calcification. In some embodiments, the disorder is calcific aortic stenosis (CAS). Generally, the methods include administering a therapeutically effective amount of an inhibitor of MAOA as described herein, to a subject who is in need of, or who has been determined to be in need of, such treatment.

### CLAIM OF PRIORITY

This application claims the benefit of U.S. Provisional Application Ser. No. 62/824,383, filed on Mar. 27, 2019. The entire contents of the foregoing are incorporated herein by reference.

### TECHNICAL FIELD

Described herein are methods for the treatment of disorders associated with aortic valve calcification. In some embodiments, the disorder is calcific aortic stenosis (CAS). Generally, the methods include administering a therapeutically effective amount of an inhibitor of MAOA as described herein, to a subject who is in need of, or who has been determined to be in need of, such treatment.

### BACKGROUND

Human aortic valve calcification with associated thickening of the valve leaflets is the leading cause of non-rheumatic aortic stenosis (1). Initially considered a degenerative pathology due to its prevalence in old age, and viewed as a biologically active process with a spectrum ranging from aortic sclerosis (2) to overt calcific aortic valve disease (CAVD) (3). An ever-expanding aging population brings along a rise in severe CAVD rates, with a combined prevalence in European countries, and North America expected to increase from 1.5 to 3.9 million by 2050 (4). Lack of early symptoms precludes early diagnosis. Left untreated severe CAVD leads to heart failure and death. Currently, no effective drug therapy is available; surgical or transcatheter aortic valve replacements remain to be the sole treatment options (5). Due to CAVD's economic and health burden, exploring non-invasive therapeutic strategies is imperative to fill this highly unmet clinical need.

### SUMMARY

The present disclosure is based on the identification of progenitor-like subpopulation of human aortic valvular interstitial cells (VICs) CD44highD29+CD59+CD73+CD45low phenotype in CAVD with pluripotential capability including osteogenic, adipogenic, and chondrogenic differentiation. By applying single-cell multi-OMICs techniques and network medicine, the VIC subpopulation was identified as a disease driver population in CAVD. Integrating proteomics and single cell transcriptomics of DDP and MSCs revealed MAOA and CTHRC1 as potential regulators of calcification. MAOA and CTHRC1 silencing inhibited calcification of human VICs. Thus, provided herein are methods for therapeutic intervention in CAVD that include administering inhibitors of MAOA.

Thus provided herein are methods for the treatment of a disorder associated with aortic valve calcification. The methods include administering a therapeutically effective amount of an inhibitor of monoamine oxidase subtype A (MAOA), to a subject who is in need of, or who has been determined to be in need of, such treatment. Also provided herein are compositions comprising an inhibitor of monoamine oxidase subtype A (MAOA), for use in the treatment of a disorder associated with aortic valve calcification in a subject who is in need of, or who has been determined to be in need of, such treatment.

In some embodiments, the methods further include identifying the subject as having a disorder associated with aortic valve calcification. In some embodiments, the subject has calcific aortic stenosis (CAS), coronary artery disease, carotid artery disease, peripheral artery disease, vein graft failure, arteriovenous fistula, and scleroderma. In some embodiments, the subject is a mammal, preferably a human.

In some embodiments, the inhibitor of MAOA is a small molecule inhibitor.

In some embodiments, the small molecule inhibitor of MAOA is selected from the group consisting of Befloxatone (MD370503); Bifemelane (Alnert, Celeport); Brofaromine (Consonar); Cimoxatone (MD 780515); Clorgyline (or Clorgiline); Methylene Blue; Minaprine (Cantor); Moclobemide (Aurorix, Manerix); Phenelzine (Nardil); Pirlindole (Pirazidol); Toloxatone (Humoryl); Tyrima (CX 157); Tranylcypromine (nonselective and irreversible, Parnate); Isocarboxazid (1-benzyl-2-(5-methyl-3-isoxazolylcarbonyl)hydrazine-isocarboxazid, Marplan, Marplon, Enerzer); Molindone; Ladostigil; VAR 10303; M30; Hydralazine; Phenelzine; quinacrine; and salts and combinations thereof.

In some embodiments, the inhibitor of MAOA is an inhibitory nucleic acid targeting MAOA. In some embodiments, the inhibitory nucleic acid targeting MAOA is an antisense RNA; antisense DNA; chimeric antisense oligonucleotide; antisense oligonucleotide comprising modified linkages; interference RNA (RNAi); short interfering RNA (siRNA); a short, hairpin RNA (shRNA); small RNA-induced gene activation (RNAa); small activating RNAs (saRNAs); gapmer; mixmer; locked nucleic acid (LNA); or peptide nucleic acid (PNA).

In some embodiments, the inhibitory nucleic acids include antisense RNA, antisense DNA, anti-sense oligonucleotides (ASO), DNA aptamers, DNA decoys, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), gapmers, mixmers, morpholino phosphoroamidates (MF) LNAs (locked nucleic acids), PNAs (peptide nucleic acids), ribozymes, circular RNAs, RNA aptamers, RNA decoys, long non-coding RNAs, CRISPR guide RNAs, small guide RNAs, antagomirs, RNA sponges, microRNA sponges, oligonucleotide tagged antibodies, plasmid-, DNA- or RNA-complexed microbubbles, DNA or RNA conjugated lipid nanoparticles, oligonucleotide conjugated nanomaterials/nanoparticles, or combinations thereof.

In some embodiments, the inhibitory nucleic acid is modified, e.g., comprises a modified backbone, preferably comprising phosphorothioates, phosphotriesters, methyl phosphonates, short chain alkyl or cycloalkyl intersugar linkages or short chain heteroatomic or heterocyclic intersugar linkages. In some embodiments, the inhibitory nucleic acid comprises one or more modified nucleotides, optionally comprising one or more modified nucleobases nucleotides modified at the 2′ position of the sugar.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. Identifying surface markers for putative disease driver population of VICs in CAVD. A. High throughput screening flow cytometry (HTS-FC) of freshly isolated VICs (n=8 CAVD patients, pooled) and MSCs cultured for 2 weeks in three different media: growth or normal media (NM), osteogenic media (OM) or adipogenic media (AM). B. Expression heatmap of VICs and MSCs (percent positive staining) of HTS-FC and corresponding first-neighbors PPI network nested within the heatmap. Blue square nodes are seeded target genes; dark circle source nodes are surface markers with high expression in all four cell conditions; light circles are first and second neighbors connecting the seeded and surface marker genes. The node size reflects the degree of hub-centrality (betweenness centrality). C. Plotting edge counts vs. betweenness centrality shows CD44 as the most overly connected node in this “calcification” network having both the highest edge count and betweenness centrality measure. D. Representative IHC staining of a calcified aortic valve leaflet. Positive marker IHC stains are pseudo-colored to facilitate visualization and quantification of markers. E. Percent positive stained cells per layer quantified based on the average on ten donor valves. (***) p<0.001. E. Histograms of the relative abundance scale of positive staining frequency of selected surface markers having high expression in all cell conditions. Two conditions (VICs and MSC-OM) with their respective isotype controls (IC) (presented as dark grey). IHC=immunohistochemistry; PPI=protein-protein interaction; MSC=mesenchymal stem cells; VICs=valvular interstitial cells.

FIGS. 2A-J. Identifying and characterizing the CD44^{high} VICs in human calcific aortic valve leaflets. A. Leaflet schematic of area that is close to calcification nodule (*). B. Tissue cell spread technique applied to specific areas containing calcification nodules, depicting the region of interest, n=4 valve donors C. Feulgen nuclear staining after tissue spread preparation showing cells maintaining relatively similar orientation as they were during pre-processing (in situ). D. CD44 immunostaining after tissue spread preparation on relatively well-preserved tissue microarchitecture showing CD44+ cells (red arrows and inset) in close proximity to calcification nodule (*). E. Immunofluorescence microscopy imaging at 60× of region near the calcification. CD44 (red) and osteocalcin (OCN) (green) co-localization (yellow) is shown along with cells containing atypical nuclear morphology. Cells with cupped nuclei (DAPI staining, yellow arrow) near calcification with CD44+ staining F. Feulgen staining of nuclei appearing as “beads in series” (left image) or as “stacked bell-shaped cups”. G. CD44+ staining and atypical “metakaryotic” nuclei either in stacked cups configuration or (H.-I.) serial configuration (yellow arrows), rendered in 3D from z-stack of confocal immunofluorescent images. J. 20× magnification showing atypically nucleated CD44+ cells.

FIGS. 3A-G. High Content Imaging Analysis of putative DDP (progenitors) VICs nuclei vs. non-progenitors. Tissue sections are stained with PE-CD44 (red) and AF488-CD29 (green) A. CD44 and CD29 stained cells near calcification area (scale=50 um). Majority of cells stain with both CD44 and CD29, representative image B. CD44-positive cells distant from calcification. None were co-staining with CD29 C. Nuclei masking (green boundary) for putative progenitors CD44+CD29+ cells and for (D.) putative non-progenitor cell, used to measure nuclear morphometry using Cell Profiler. E. Area of nuclei comparison F. Compactness on the nuclei comparison. G. Eccentricity of the nuclei comparison. n=4 donors per group. Statistical testing done with Wilcox non-parametric rank sum.

FIGS. 4A-F. Identifying the disease driver population in CAVD. A. Representative image of FACS sorted aortic VICs cultured in either normal media (NM), high phosphate media (PM), or osteogenic media (OM). Unsorted VICs, sorted CD44^{high}, and sorted CD44+ cells calcified under OM and PM conditions (PM>OM), but not in NM, Alizarin Red staining at day 21 (n=3). B. Multi-color flow cytometry shows co-localization of CD44^{high} VICs with CD29, CD59, and CD73 positive staining, but with lesser staining of CD45, n=3 donors. C. Immunohistochemistry of areas proximal to calcification show more abundant staining of markers CD44, CD29, CD59, and CD73 than in areas more distal to the calcification (n=3) D. Sorted VICs population CD44^{high}CD29⁺CD59⁺CD73⁺CD45^{low} cells readily differentiated into calcifying osteoblast-like cells in OM (Alizarin Red staining) and fat globule containing adipocyte-like cells in AM (Oil Red O staining) similar to MSCs differentiation in these specialized media. Alcian blue staining showing chondrocyte-like DDP VICs in CM and mixed (MIX) unsorted aortic VICs show no comparable differentiation in OM, CM or AM. Representative images, n=4 donors. E. Comparison of relative expression (expression intensity; warm/red=high to cool/blue=low scale) of ALPL (a marker of early calcification), CD44, CD29 and Ki67 between calcified and non-calcified portions of valves with mass cytometry, n=3 donors. F. SPADEs of calcified leaflets contained more cells (bigger sized node circles) with higher expression intensities when compared to non-calcified SPADEs, shown through larger nodes with warmer colors versus smaller nodes with cooler colors (see insets).

FIGS. 5A-G: Single cell RNA sequencing, scRNA-seq, of in vitro 2-week calcification assay with MSC-NM, MSC-OM, MIX-NM, MIX-OM, PUR-NM, and DDP-OM cells. A. Schema of single cell InDrops scRNA-seq of in vitro calcification assay. MSCs and VICs are cultured in either OM or NM for 2 weeks. Cells were quickly detached and immediately sorted using InDrops following single cell cDNA library synthesis and sequencing (Illumina NextSeq). B. tSNE plot showing k means clusters (k clust) of MSCs and how they separate. C. tSNE plot calculated with k-means clustering information showing MSC-NM and MSC-OM separation. D. tSNE plot calculated with k-means clustering information showing VICs: MIX-NM, MIX-OM, PUR-NM, and PUR-OM separation. E. tSNE plot showing k means clusters (k clust) of VICs and how MIX-NM, MIX-OM, DDP-NM, and DDP-OM separate. F. MSC k-clusters expression heatmap of increased DGEs from calculated fold change ≥2.0 of k4 vs k2 cluster at FDR, q-value ≤0.05. G. VICs k-clusters expression heatmap of increased DGEs from calculated fold change ≥1.5 of k4 vs (k1+k2) clusters at FDR, q-value ≤0.05. DGE=differentially expressed genes, FDR=false discovery rate, NM=normal media, OM=osteogenic media, MSCs=mesenchymal stem cells, VICs=valvular interstitial cells, QC=quality control, tSNE=t-distributed stochastic neighbors embedding, MIX=mixed unsorted VICs, DDP=sorted CD44^{high}CD29⁺CD59⁺CD45^{low} VICs.

FIGS. 6A-J. In vitro time-course proteomic profiling. A. Experimental setup of MSC, VICs MIX, and DDP grew in culture using growth media then transitioned into either in OM or NM conditioned media. Time course starting from exposure, collecting proteins at different timepoints in weekly intervals, from 0 weeks to 3 weeks post OM or NM switch: days 0, 7, 14, and 21. B. Alizarin red calcification assay after OM treatment at 14 days and 21 days, comparing MSCs with DDP and MIX (n=4). The phenotype of DDP closely resembles MSC in OM behavior. C. Total proteins identified from proteomics in MSCs and VICs set up with a large portion of shared proteins. D. Principal component analysis, PCA, of each sample-condition depicting proximity of MSC-NM and MSC-OM at 0 weeks or immediately after switching to condition media. With increasing time points, the separation distance between temporally paired OM and NM conditions, increase in the 2D PCA space. E. Heatmap of MSC dataset showing the differences in proteome abundances between media conditions are higher in the time points d14 and d21 than in d0 and d7. F. Analysis workflow of MSC-OM and MSC-NM proteomics data. From the global proteome, sample conditions were analyzed two ways: a rank-regression across time points and a two-group comparison between media conditions. The resulting differentially expressed proteins (DEP) were consolidated to and designated as DEPs for MSC. G. DEP in MSC-OM that increases over time (86 proteins). H. Analysis workflow of VIC-MIX and VIC-DDP proteomics data. From the global proteome of either VIC-DDP or VIC-MIX, sample conditions were analyzed two ways: a rank-regression across time points and a two-group comparison between media conditions. The resulting differentially expressed proteins (DEP) were consolidated to and designated as DEPs for VICs DDP or DEPs for VICs MIX. Further comparison of these 2 DEP lists reveals DDP-DEP specific proteins unique to DDP not identified in MIX cells. I. Increasing DEP for VICs DDP specific proteins (71 proteins) J. Overlap between the DEP for MSC (86) and the DEP-DDP specific (71) with 2 shared proteins.

FIGS. 7A-G. Intersection between Transcriptomics and Proteomics Data. A. 13 proteins appear at least twice in the increasing DEP or DEG lists from either proteomics or single cell transcriptomics analysis among OM conditioned MSCs and VIC-DDP cells. CTHRC1 is the only gene present in three increased differential expression lists. MAOA is an enzyme that has never been associated with aortic valve calcification. B. Silencing of MAOA and CTHRC1 mRNA in VICs then cultured in NM or OM showed statistically significant reduction in expression of genes after 10 days in culture. C. VIC intracellular tissue non-specific alkaline phosphatase (ALPL) activity (after 10 days of culture) correspondingly decrease in OM-treated MAOA, or CTHRC1 silenced VICs, n=3 donors. D. Osteocalcin mRNA expression is also decreased upon MAOA or CTHRC1 silencing after 10 days in culture E. Alizarin red staining of VICs after 21 days grown OM with either PBS (control) or moclobemide 10 μM or pirindole 10 μM. (n=3) F. IHC of MAOA-positive cells showing proximity to calcification areas, but not in cells distal to calcification. G. IHC of CTHRC1-positive cells showing proximity to calcification areas, but not in cells distal to calcification. Representative images, n=3 donors. For FIG. 6A-D: ns p>0.05, (*) p≤0.05, (**) p≤0.01, (***) p≤0.001. For FIG. 6E-F: (*)=calcification area

FIG. 8. Protein-protein interaction (PPI) Network PPI network connecting the subnetworks of four DDP markers (blue, inner circle) and their first neighbors (blue, outer circle), sc-transcriptomics and proteomics filtered targets (yellow, inner circle) and their first neighbors (yellow, outer circle), and aortic valve and vascular calcification associated markers (red, inner circle) and their first neighbors (red, outer circle). Overlapping proteins are present between the respective subnetworks.

### DETAILED DESCRIPTION

Molecular regulatory networks in CAVD have been identified by multi-OMIC approaches on a layer and disease-stage basis (6). The predominant extracellular matrix composition of each of the aortic valve layers is essential for the proper valve function: collagen (fibrosa), proteoglycans (spongiosa), and elastin (ventricularis) (7). Endothelial cells pave the valvular luminal surface, while valvular interstitial cells (VICs) comprise the majority of the valve interstitium. VICs in normal valve development function primarily by coordinating matrix organization and compartmentalization (8). The current paradigm suggests that VICs are a plastic cell population: in disease conditions, VICs differentiate into activated collagen-producing myofibroblasts contributing to matrix remodeling and fibrosis (9). However, whether a subpopulation of VICs also initiates the calcification process is unclear. Inspired by the emerging concept of valvular cells heterogeneity (6, 10, 11), we sought to determine whether such an osteogenic sub-population exists in human CAVD. These investigations are difficult in human tissues; tools have been lacking and limited to histopathological studies. Described herein are studies that used a combination of high throughput profiling technologies to test the hypothesis that a specific disease driver population (DDP) of aortic VICs present in human CAVD acts as progenitors capable of osteogenic differentiation to promote calcification (12-14).

This question was addressed by employing multiple omics and single-cell methods in a tiered workflow. As shown herein, a pluripotent progenitor-like CD44^{high}CD29⁺CD59⁺CD45^{low} VIC population was comprehensively characterized and mapped to nodes of calcification located within the disease-prone fibrosa layer. As shown herein, CAVD leaflets contained cells with an MSC-like “metakaryotic” nuclear phenotype in 3D (tissue spread preparation) (15) and 2D (immunofluorescence). HTS-FC and network analysis demonstrated CD44 taking a central role in DDP-VIC regulation of calcification, as shown in vitro. These results corroborate other reports on CD44 as a key molecule in vascular calcification and VIC activation (16, 17). However, CD44 is ubiquitously expressed in many tissues, including leukocytes, fibroblasts, and the endothelium (18), and so therapeutic targeting of CD44 may have unintended systemic effects. CD29 was recently reported as a procalcifying progenitor cells marker in atherosclerosis (19), while CD59 is highly associated with adipogenic and osteogenic capacities of mesenchymal stromal cells (20); and CD73 is implicated in innate immunity (21), thus deprioritizing them as potential CAVD therapeutic targets even though we acknowledge CD73's role in valvular mineralization as have been reported recently (22-24).

In establishing the CD44^{high}D29⁺CD59⁺CD73⁺CD45^{low} phenotype as a DDP-VIC in CAVD, gene expression was explored through time-course proteomics and single-cell transcriptomics (scRNA-seq) analyses, comparing valve FACS isolated and in vitro expanded CD44^{high}CD29⁺CD59⁺CD73⁺CD45^{low} DDP-VICs against its autologous source of unsorted MIX-VICs. In scRNA-seq, a subcluster of MSC-OM cells expressing various collagens (e.g., COL1A1, COL1A2, COL3A1, COL5A1, COL5A2, COL6A1) and calcification-related genes (e.g., SPARC/osteonectin, SPOCK1, TNFRSF11B/osteoprotegerin) was found, confirming that these progenitors are undergoing active fibroblastic and osteoblastic differentiation in response to osteogenic stimuli (25). As this phenomenon is not seen in VICs stimulated with OM, it is unclear whether these genes could be appropriate for therapeutic consideration in CAVD (26).

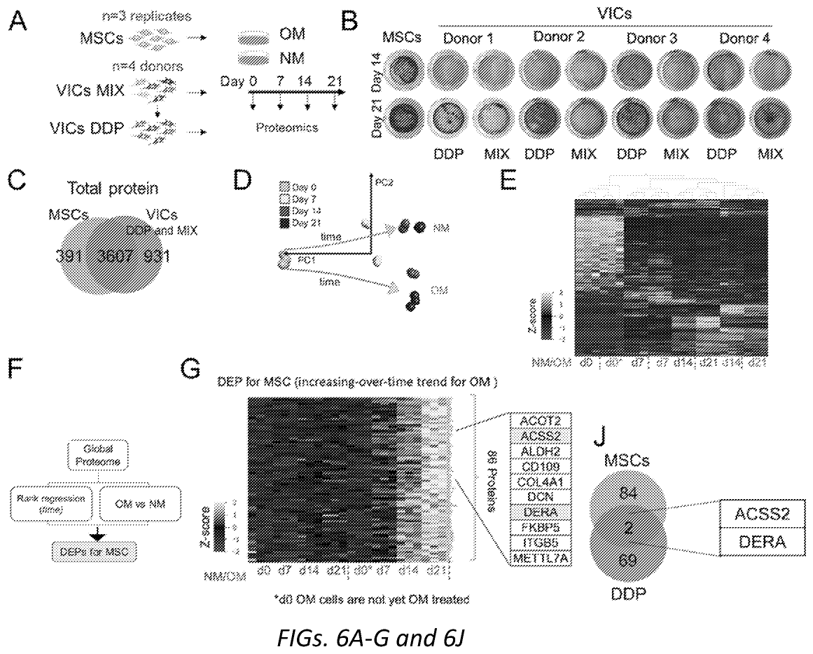

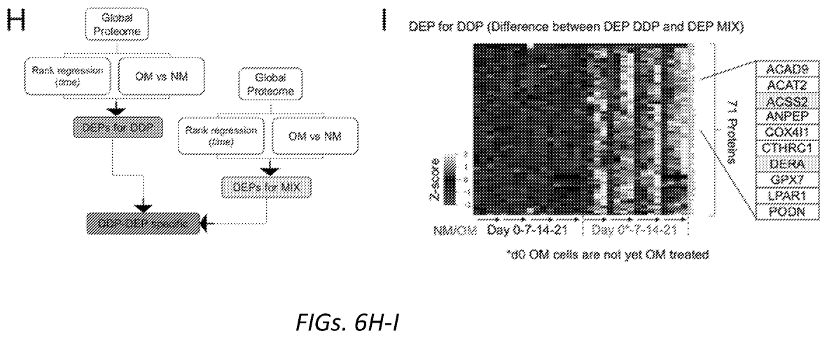

A majority of DDP-VICs that comprise the scRNA-seq k4 cluster have CD44^{high}CD29⁺CD59⁺CD45^{low} procalcific phenotype. Genes increased in DDP-VIC k4 cluster versus other VIC-OM clusters (k3, k5, and k6) include stem cell markers—CD34, TMTC1, COL8A1, FKBP5, ANPEP, TGF-β receptor 2 (TGFBR2), PLXNA2, ELN, CTHRC1, and MAOA, among others. Consistently, MAOA, TMTC1, and COL8A1 were also enriched in the time-course MSC-OM proteomics, while CTHRC1 and ANPEP are enriched in the VIC DDP-OM proteomics time-course. Enrichment of CD34 mRNA, congruent with the initial HTS-FC data and VIC time-course proteomics (DDP-OM), reflected the progenitor cell-like nature of the DDP-VICs. Increased TGFBR2 and PLXNA2 expression suggested osteoblastic potential in this subset (k4) of CD44^{high}D29⁺CD59⁺CD73⁺CD45^{low} DDP-VICs. PLXNA2 mediates osteoblastic differentiation in bone mesenchymal progenitors via RUNX2 regulation (27), similar to TGFBR2 (28). Other overlaps in gene expression were COL4A1, CRLF1, and DCN differentially enriched in both MSC-OM scRNA-seq and MSC-OM proteomics and ACADSB enriched in both VIC DDP-OM proteomics and MSC-OM proteomics (FIG. 6A). Lastly, FKBP5, a glucocorticoid pathway-binding protein that is present in VIC DDP-OM proteomics, DDP-VIC scRNA-seq, and MSC-OM proteomics, has yet unclear implications in the calcification process.

MAOA and CTHRC1 could serve as potential novel therapeutic anti-calcification targets based on their co-regulated induction with other calcification related genes like SPARC, ELN, FN1, FBN, and COL8A1 as seen in our scRNA-seq data. MAOA is also co-enriched with CD34, a stem cell marker, and various myofibroblast and osteoblast differentiation markers, including ELN, COL8A, ACTA2, and TGFBR2. Pathways enrichment validates this shared mechanism of osteoblast differentiation of progenitor-like cells between the VIC and the MSC scRNA-seq datasets, corroborating that the CD44^{high}D29⁺CD59⁺CD45^{low} DDP-VICs is indeed a newly identified disease drivers in human CAVD.

Studies in the past showed that high serotonin levels could cause oxidative stress in human heart valves (29). MAOA, however, has not been reported as a regulator in the calcification process. Herein, MAOA silencing during VIC osteogenic differentiation effectively reduced calcification-related gene expression and intracellular alkaline phosphatase activity, suggesting a novel amine oxidation perturbation pathway interrupting calcification. CTHRC1 is implicated in CAVD through bioinformatic screening studies (30) of human aortic valve gene expression datasets. CTHRC1 also binds and activates WNT5A, and WNT11(31), members of the non-canonical WNT signaling in human CAVD (32). Studies reported CTHRC1 present in calcifying human atherosclerotic plaques (33). The present study showed in vitro that CTHRC1 loss-of-function led to a reduction in calcification-related genes as well as alkaline phosphatase activity in VICs subjected to OM culture conditions, thereby confirming CTHRC1 as potential anti-calcification target.

In conclusion, using a stepwise enrichment of pro-calcifying cell subpopulations, we isolated a novel disease driver VIC population with a CD44^{high}D29⁺CD59⁺CD45^{low} pro-osteogenic progenitor-like phenotype. By employing single cell analytic tools, transcriptomics and proteomics methods, and network analysis, a roster of potential therapeutic candidates for human CAVD was identified. Provided herein are methods of pharmacotherapy for CAVD that target these disease-associated genes.

Methods of Treatment

The methods described herein include methods for the treatment of disorders associated with aortic valve calcification. In some embodiments, the disorder is calcific aortic stenosis (CAS)(34); Lindman et al., Nat Rev Dis Primers. 2016 Mar. 3; 2:16006; coronary artery disease; carotid artery disease; peripheral artery disease; vein graft failure; arteriovenous fistula; and scleroderma. Generally, the methods include administering a therapeutically effective amount of an inhibitor of MAOA as described herein, to a subject who is in need of, or who has been determined to be in need of, such treatment.

As used in this context, to “treat” means to ameliorate at least one symptom of the disorder associated with aortic valve calcification. Often, CAS results in symptoms including dyspnea due to heart failure, angina, syncope, and dizziness; thus, treatment can result in a reduction in one or more of these symptoms. Heart failure due to CAS results in deaths in many cases. MAOA inhibition may ameliorate the development of heart failure and may thus reduce the motility of CAS patients. Administration of a therapeutically effective amount of a compound described herein for the treatment of a condition associated with aortic valve calcification will result in decreased levels of aortic valve calcification.

Calcification participates in the pathogenesis and clinical complications of other disorders such as coronary artery disease, heart attack, carotid artery disease, peripheral artery disease, vein graft failure and arteriovenous fistula failure. MAOA inhibition may ameliorate these problems.

In some embodiments, the methods include identifying a subject who has aortic valve calcification. One of skill in the art would readily be able to identify or diagnose a subject who has aortic valve calcification using methods known in the art; for example, a diagnosis of AS can be established using an echocardiographic exam(34); see, e.g., Lindman et al., Nat Rev Dis Primers. 2016 Mar. 3; 2:16006.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans more accurately. Levels in plasma may be measured, for example, by high performance liquid chromatography.

Inhibitors of MAOA

The present methods include the administration of MAOA inhibitors. A number of MAOA inhibitors are known in the art, including small molecules and inhibitory nucleic acids.

Small Molecule Inhibitors of MAOA

Small molecule inhibitors of MAOA include Befloxatone (MD370503); Bifemelane (Alnert, Celeport); Brofaromine (Consonar); Cimoxatone (MD 780515); Clorgyline (or Clorgiline); Methylene Blue; Minaprine (Cantor); Moclobemide (Aurorix, Manerix); Phenelzine (Nardil); Pirlindole (Pirazidol); Toloxatone (Humoryl); Tyrima (CX 157); Tranylcypromine (nonselective and irreversible, Parnate); Isocarboxazid (1-benzyl-2-(5-methyl-3-isoxazolylcarbonyl)hydrazine-isocarboxazid, Marplan, Marplon, Enerzer); Molindone; Ladostigil; VAR 10303; M30; Hydralazine; Phenelzine; quinacrine; and salts and combinations thereof. See, e.g., Dhiman et al., Molecules 2019, 24(3), 418.

Inhibitory Nucleic Acids

Inhibitory nucleic acids useful in the present methods and compositions include antisense oligonucleotides, siRNA compounds, single- or double-stranded RNA interference (RNAi) compounds such as siRNA compounds, modified bases/locked nucleic acids (LNAs), peptide nucleic acids (PNAs), ribozymes, and other oligomeric compounds or oligonucleotide mimetics that hybridize to at least a portion of the target MAOA nucleic acid and modulate its function. Exemplary sequences of human MAOA are as follows:

| | | amine oxidase |
|---|---|---|
| | | [flavin-containing] |
| Nucleic Acid | Amino Acid | A Isoform |
| NM_000240.4 | NP_000231.1 | isoform 1 |
| NM_001270458.1 | NP_001257387.1 | isoform 2 |

In some embodiments, the inhibitory nucleic acids include antisense RNA, antisense DNA, anti-sense oligonucleotides (ASO), DNA aptamers, DNA decoys, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), gapmers, mixmers, morpholino phosphoroamidates (MF) LNAs (locked nucleic acids), PNAs (peptide nucleic acids), ribozymes, circular RNAs, RNA aptamers, RNA decoys, long non-coding RNAs, CRISPR guide RNAs, small guide RNAs, antagomirs, RNA sponges, microRNA sponges, oligonucleotide tagged antibodies, plasmid-, DNA- or RNA-complexed microbubbles, DNA or RNA conjugated lipid nanoparticles, oligonucleotide conjugated nanomaterials/nanoparticles, or combinations thereof.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of an RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the MAOA sequence. For example, a specific functional region can be targeted, e.g., a region comprising a known motif (i.e., a region complementary to a promoter). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of an RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of an RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general, the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

In addition, inhibitory nucleic acids targeting MAOA are commercially available, e.g., from Sigma Aldrich; origene; dharmacon; and Santa Cruz Biotechnology.

siRNA/shRNA

In some embodiments, the nucleic acid sequence that is complementary to a target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc NatlAcadSci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; FLuiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Orom et al., Gene. 2006 May 10; 372:137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NHO—CH₂, CH,˜N(CH₃)˜O˜CH₂ (known as a methylene(methylimino) or MMI backbone), CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃OCH₃, OCH₃O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, Helv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for the desired activity. Any recombinant expression system can be used, including e.g., in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y (1993).

Pharmaceutical Compositions and Methods of Administration

The methods described herein include the use of pharmaceutical compositions comprising one or more MAOA inhibitors as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Carriers that can be used include viral particles; inactivated viral particle; parts of a viral particle; specific viral proteins; ultrasound microbubble; gas filled ultrasound microbubble; lipid nanoparticle; lipid microparticle; iron compound nanoparticle; magnetic nanoparticles; other nanomaterials; and nanotubes and other nanoparticles. Supplementary active compounds can also be incorporated into the compositions.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, sublingual, conjunctival and rectal administration or localized administration due to a drug-eluting stent or other surgical procedures on the heart. Local administration may be used in atherosclerotic arteries or veins, including but not limited to the coronary artery, carotid artery, peripheral artery, vein graft and arteriovenous fistula.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for a parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

More localized delivery of the compounds can be achieved using a drug eluting stent. Stents are implants configured for use in a coronary artery, are preferably capable of radial expansion, and include a coating layer that encapsulates and provides for extended release of a compound described herein. The stent can include a polymeric or non-polymeric coating layer. See, e.g., WO 99/07308; U.S. Pat. Nos. 9,012,506; 6,258,121; 6,171,609; 6,159,488; 6,099,562; 5,873,904; 5,342,348; 5,873,904; 5,707,385; 5,824,048; 5,527,337; 5,306,286; 5,288,711; 6,153,252; and 6,013,853; and US 20190388210, among others.

The pharmaceutical compositions can be included in a container, pack, or dispenser together with instructions for administration.

### EXAMPLES

The invention is further described in the following examples, which do not limit the scope of the invention described in the claims.

Methods

The following methods were used in the Examples below.

Approval of Study Protocol

To identify subpopulations of valvular interstitial cells (VICs) and their involvement in the origin of human calcific aortic valve disease (CAVD), we analyzed aortic valve leaflets obtained during aortic valve replacement surgical procedures on patients with CAVD. All patients provided written consent and the study protocol for the collection of human samples was approved by the Institutional Review Board and the Human Research Committee of Brigham and Women's Hospital. Detailed review of the clinical summaries revealed that there were no significant differences between the patients for risk factors predisposing for CAVD such as coronary artery disease or other conditions. This study used a total of 33 human aortic valves composed of 23 male and ten female patients with a mean age of 67±10 years old.

Cell Culture Maintenance

Culture media used for in vitro experiments are the following: (1) serum-free media is Dulbecco's modified eagle medium, DMEM (11965-092, Thermo Fisher Scientific) with 1% penicillin/streptomycin (P/S) (2) normal media (NM) is DMEM with 5% of fetal bovine serum, FBS (10082147, ThermoFisher Scientific). (3) growth media (GM), is DMEM with 10% FBS. Osteogenic media (OM) was prepared using 10 mM β-glycerophosphate (35675-50GM, EMD Millipore), 0.01 mM dexamethasone (ICN19456125, Fisher Scientific) and 0.05 mg/mL ascorbic acid (A4544-25G, Sigma-Aldrich) in NM. Chondrogenic media was prepared using 0.1 μM dexamethasone, 1× Insulin-Transferrin-Selenium-Ethanolamine (ITS+) (51500056, Thermo Fisher Scientific)), 1 mM of 2-phospho-L-ascorbic-acid (49752-10G, Sigma-Aldrich), 1 mM of L-Proline (P5607-25G, Sigma-Aldrich) and 0.1% of TGF-β1 (100-B-001/CF, R&D) in serum-free NM. Adipogenic media contains 1 μM dexamethasone, 0.125% of human insulin solution (I-9278, Sigma-Aldrich), 0.5 mM 3-isobutyl-1-methylxanthine, IBMX (I5879, Sigma-Aldrich) and 60 μM of indomethacin was added to NM. For initial growth of adipose tissue derived human MSC cell-lines (ATCC), PeproGrow™ hMSC Mesenchymal Stem Cell Media (Peprotech) was used. For all washing steps, if not indicated otherwise, autoclaved phosphate buffer saline (PBS) was used.

Isolation of Aortic VICs

A 1 to 5 mm thick leaflet tissue containing contiguous parts from the leaflet base to tip including calcified nodules was obtained from each donor valve leaflet for histology prior to processing for enzymatic digestion. Processing the aortic valve tissue required manually removing large calcific nodules (˜≥2 mm diameter) from the tissue using fine forceps and mincing the tissue into ≤1 mm³ pieces. Prior to mincing, if the specimen was going to be processed downstream for mass cytometry (CyTOF) or single cell RNA sequencing, we cut valves into halves with one half containing all the visible calcific nodules, designated as the “calcified” sample of the valve, and one half visibly calcification-free, designated as the “non-calcified” sample. For specimens undergoing in vitro culturing for further experiments including FACS, high-throughput surface markers flow cytometry screening and multi-color flow cytometry, we minced valves without separating calcified and non-calcified portions. VICs were released from the minced tissue by enzymatic digestion in 37° C. using ˜6 mL sterile filtered solution of 1 mg/mL collagenase IA-S(Sigma) prepared in DMEM with 10 mM HEPES buffer and 1% penicillin/streptomycin supplementation, for one hour with inverted mixing every 20 min. The content was then vortexed, and after the pieces had settled, the media was aspirated. After another washing step with basal media DMEM, we added a fresh filtered collagenase solution to the tissue and incubated it for another 3 hours. Enzymatic digestion was stopped using ˜600 μl of 100 FBS. The cell suspension was then filtered through a 100 μm cell strainer (BD Biosciences), washed, and cultured into 6 cm or 10 cm diameter Petri dishes or re-suspended in 3% FBS in DMEM with 1.0 mM HEPES for downstream flow cytometry or CyTOF experiments.

Immunohistochemistry (IHC) of Aortic Valves

We conducted quantitative immunohistochemistry of cryosections through the aortic valve leaflet (n=10). Samples frozen in OCT compound (Ager Scientific) were cut consecutively into 6 μm histological sections. After fixation (4% paraformaldehyde), peroxidase blocking (0.3% hydrogen peroxide), and serum blocking (4% fetal calf serum), sections were incubated with primary antibodies (Antibody supplementary table) associated with calcification, VICs or mesenchymal stem cell (MSC) properties. After 1.5 hours of incubation and an additional washing step, we incubated the sections in a biotin-labeled secondary antibody at a concentration of 1:100, followed by a streptavidin-peroxidase treatment and an AEC solution (Thermo Fisher) incubation to achieve color development. After a quick counterstain with Harris-hematoxylin (Thermo Fisher), we examined the slides using an Omnyx VL4 scanner (GE Healthcare), then processed them with the Elements software version 3.20.

Measuring Staining Positivity Over Three Layers in IHC Using Python Sci-Kit

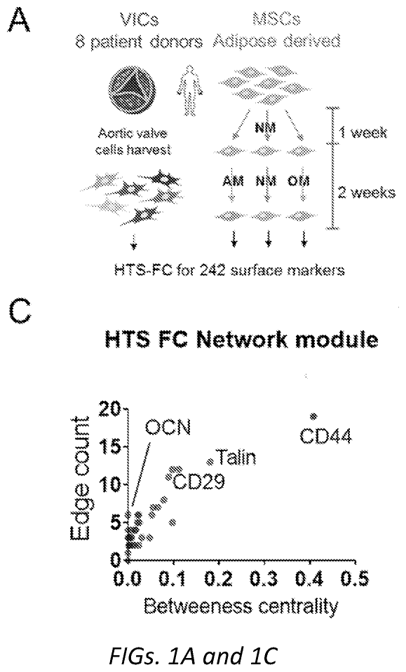

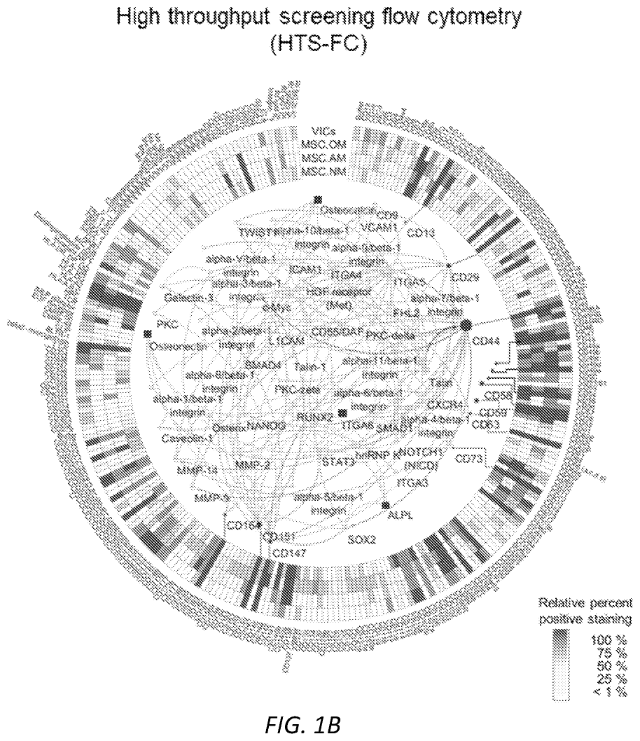

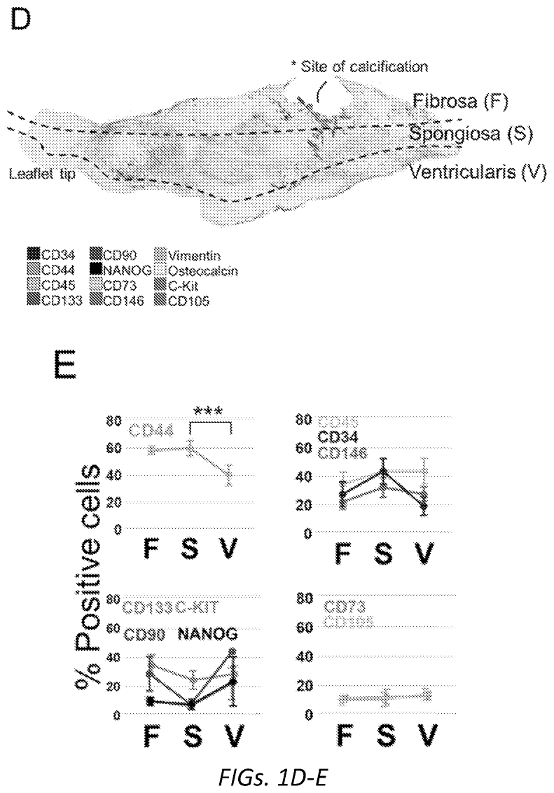

For ten aortic valve leaflets, unbiased cell counting and quantification of positive immunostaining signals per cell for the following markers listed in FIG. 1 were done using sci-kit-image for image processing (Python). Images scanned from Omnyx VL4 scanner (GE Healthcare) were exported at full resolution (highest resolution at 40× or 0.019 micrometer/pixel) and were then exported as a large scan of the whole valve (11508×3273 pixels²). The large image scan was split into manageable sizes (˜2300×1600 pixels). Each split image was processed for color deconvolution to clearly distinguish the blue hematoxylin staining against the brown DAB staining. Then, image colors were inverted so that signal stains (nuclear and IHC markers) could appear bright against the dark background (negative staining stroma). Because calcification areas and crystals stain similarly with the hematoxylin as the nuclei, calcification nodules were excluded from the analysis by thresholding each object/particle identified by the algorithm using object largest diameters from blob sampling. For example, if the hematoxylin staining blobs are detected in close apposition (adjacent) with each other, the diameters are cumulatively summated and will exceed threshold diameter in micrometers. None of the true cell nuclei, which are each separated from other adjacent cell nuclei by non-hematoxylin staining cytoplasm, exceed ˜20 micrometers and none of the calcification nodules staining with hematoxylin are smaller than 50 micrometers. Hence, by thresholding blob diameters, calcification areas can be excluded. After calcification exclusion, we again used blob detection to identify the nucleus of each cell using the Laplacian of Gaussian method (scikit-image.org/docs/dev/api/skimage.feature.html #id55). Cells were counted and recorded automatically to yield total cell count for the entire section. Then, blob detection of IHC signals (DAB brown color) was done with the criteria that they should overlap or be in very close proximity (≤5 um) of the identified nuclear blobs to designate it as positively staining cell. A positive DAB brown color is defined as a color hue intensity of 0.4 and above (DAB threshold≥0.4) in a range of 0 to 1.0 with 0 being white and 1.0 being the darkest brown intensity found in the image scanned as determined by the algorithm during color deconvolution step. Subsequently, each of the split images and their image enumerated cytometry data were concatenated and consolidated. Regions for fast image analysis (FRIA) for each layer were identified and the samples were quantified for percent positive staining per total cells per FRIA. The total number of positive staining cells were divided by the total number of cells to get the percent positive cells measure.

Immunocytochemistry of VICs

VICs from mixed and isolated populations were cultured to semi-confluency in separate 4 cm² chambers. Cells were washed three times before fixation with 1.0 mL of 4% paraformaldehyde (PFA) for 10 min. After discarding the PFA and washing with PBS, we added 2× permeabilization buffer (eBioscience Fix and Perm set Cat #88-8824-00) mixed in FluoroBrite™ DMEM (Thermo Fisher Cat #A1896701) for 10 min to ensure permeabilization. After washing again, we added 1× permeabilization buffer, 1% PermWash (BD Biosciences) in FluoroBrite DMEM solution, and 1:100 primary antibodies to the cells for 30 min in room temperature in the dark, while ensuring a gentle shaking. After 2× gentle washing in PBS, the secondary antibody cocktail was prepared with matching specificity to the primary antibody cocktail, at 1:500 concentration of the antibody. After 30 min of incubation in the dark, slides were gently washed 3× with PBS. The slides were then stained with Hoechst 33543 (Biotium), 1:100 in FluoroBrite DMEM for 10 minutes. After 2× washing with pure FluoroBrite™ DMEM, imaging was done with Nikon Eclipse Ti A1 Confocal microscope in 20× and 60× water immersion magnification.

High Throughput Screening (HTS) of 242 Surface Cellular Markers in Aortic VICs and MSCs

VICs of calcified aortic valves and adipose stroma derived MSCs from a cell-line (ATCC® PCS-500-011™, LOT #80622175) after differentiating in NM, OM and AM media condition for 2 weeks, underwent high throughput screening flow cytometry (HTS-FC). To release single valvular cells, we conducted valve tissue digestion as described above. Clumps, debris, and calcium crystals were removed through washing and cell straining (Corning, cat #431752). The cells for each donor valve (n=8) were pooled and counted separately. Each cell suspension was adjusted to a concentration of 1×10⁶ cells per mL in a 22 mL volume in order to dispense 250,000 cells per well to the 242 unique single antibody surface marker-per-well kit (BD Bioscience Lyoplate Human Cell Surface Marker Screening Panel). MSCs were passaged with Accutase (BD Biosciences) and expanded to 18 150 cm2 flasks, with 6 flasks per media condition. Cells from culture and cells directly coming from valves were then subjected to a standard FACS protocol with antibody staining followed by APC-conjugated antibody staining with stringent cell washes in between. They were then fixed with 4% PFA before flow cytometric reading. A BD FACS Canto II with 96-well plate high throughput sampler (HTS) at the Dana-Farber Cancer Institute Flow Cytometry Core was used and data was analyzed using Cytobank and FlowJo 10.7 software. To compare relative surface marker expression per sample condition, we calculated the percentile rank from percent positive staining measurements. We then took the 85^{th} percentile as cut-off for categorically labeling that specific CD(n) marker as highly expressed in each of the sample conditions (VICs, MSC-NM, MSC-OM, MSC-AM).

Network Analysis of Consolidated FACS HTS Data with PPI Network

13 markers were used as source nodes to construct a directed interactome network with MetaCore™ (Clarivate). The markers were connected with shortest paths and a maximum of 2 link nodes in the paths. The network was pointed to a calcification related context by seeding osteocalcin, tissue-nonspecific alkaline phosphatase (ALPL), and osteopontin as markers for calcification as end-target nodes. Additionally, the hub and degree centrality were measured using Cytoscape version 3.6, and Gephi version 0.9.1

3-Dimensional Tissue Cell Spread Immunohistochemistry (IHC)

We used fresh, aortic valve tissues that were not dried excessively or cooled down below room temperature (n=2). For the Carnoy's fixative-based method, we prepared 3 parts of ethanol in 4° C. (or on ice) and 1 part of glacial acetic acid at room temperature. We submerged the valve leaflet (calcified valve) in 45 mL of Carnoy's fixative for 30 minutes. After fixation for 3-4 hours, the fixative was washed off with 70% ethanol using fives changes of fresh ethanol solution. Fixed valves in 70% ethanol could be stored at 4° C. for a year without losing quality of IHC or immunofluorescence (IF) staining quality.

A piece of tissue was pre-warmed to 37° C. solution then placed in a Coplin jar containing 1 N HCl warmed at 60° C. for 8 minutes to allow for acid tissue digestion. We then rinsed off the tissue using MilliQ water (Millipore) and placed it into a fresh Petri dish containing 45% acetic acid in MilliQ water. After 15 minutes of incubation, the cells were dispersed into a single cell layer on a microscope slide. To disperse the cells, a 0.5 mm×0.5 mm×0.5 mm tissue digest was placed on top of a droplet dome of 45% acetic acid solution (˜5.0 microliter volume) in the middle of a +/+Superfrost glass slide. On top of the tissue, we placed a 22 mm×22 mm glass coverslip (#1.5) and gently applied pressure onto the coverslip using fine forceps to spread out the cells. The spreading was monitored by microscope to ensure the cells were not flattened. Once the cells were spread to a single layer, the glass slide was immediately frozen on dry ice and the coverslip was gently removed with a razor blade. The glass slide was then ready for immunohistochemistry processing as described previously.

Calcification Assay of FACS Sorted CD44+ VICs

Early passage VICs (P1-P4) from cell culture were sorted in Flow Cytometry base on their CD44 positivity and divided into three groups: CD44⁻, CD44⁺ and CD44^{high}. The three groups were then plated and grown until confluent. Osteogenic differentiation assays were conducted for three weeks after either OM or NM treatment. We used Alizarin Red (AR) 2% (Fisher Scientific) for osteogenic assays. Cells plated in 48 or 96 well plates were washed with PBS and fixed with 10% formalin for 15 min in room temperature. After washing with Milli-Q water, we added the stain and incubated for 20 min. After washing 3× with Milli-Q water, we took pictures on an iPhone7 (Apple) and quantified the amount of dye released by spectrophotometry at 550 nm.

Mass Cytometry Time of Flight (CyTOF)-Based Protein Profiling of Aortic VICs

CyTOF uses rare metal isotope-conjugated antibodies to detect and quantify target proteins. Aortic VICs prepared from collagenase digestion of the tissue recovered overnight in culture (37° C., 5% CO2 incubator) using DMEM with 10% FBS, 1× antibiotic/antimycotic and 10 mM HEPES. After the overnight culture, the cells were composed of a non-adherent majority and less than 5% loosely adherent cells. We harvested the cells and washed them with barium-free PBS (Invitrogen), then pelleted them in a centrifuge at 300 g for 10 minutes. We stained the cells for viability for 5 minutes at room temperature using Cell-ID™ Cisplatin at 5.0 μM concentration in barium-free PBS with cell density at <1×10⁷ cells/mL in a 250 μL reaction volume. The viability staining reaction was stopped using 5× cell suspension volume of MaxPar® cell staining buffer (Fluidigm). All onward steps were carried out using barium-free buffers, reagents and containers. We again pelleted the cells and resuspended them in 100 μL reaction volume at a concentration of 1.5-3.0×10⁶ cells/mL before fixing with 200 μL Fix I buffer (Fluidigm) for 10 minutes at 4° C. Fixation was stopped by washing the cells with 800 μL cell staining buffer (CSB) (Fluidigm). We permeabilized the cells using saponin-containing CSB-S buffer (Fluidigm) for 1 minute and pelleted them. Next, we incubated the cells with 500 μL nuclear antigen staining buffer (Fluidigm) for 30 minutes before washing with nuclear antigen staining perm solution (Fluidigm). The resulting cells were then pelleted at 800 g for 5 minutes and resuspended in 10 μL of the residual volume. Nonspecific antibody binding was stopped using 10 μL of 1:100 CSB-S buffer dilution of Human Fc Block (BD Biosciences) for 10 minutes at room temperature. Following the blocking procedure, we incubated the cells for 30 minutes in room temperature (with occasional agitation of the cell suspension) with 20 μL antibody cocktail. In addition to the Antibodies used for FACS as described above, we included eight additional surface antibodies and 14 intracellular marker antibodies. The cocktail was prepared in CSB-S buffer to permeate intracellularly. The antibody binding reaction was stopped by washing the cell suspension with 800 μL of CSB-S and pelleting the cells at 800 g for 5 minutes. After most of the supernatant was discarded, pelleted cells were again resuspended in 50 μL of the residual volume. Cells were all washed 2× in 800 μL and pelleted at 800 g for 5 minutes. Nuclear content interaction and bead spiking was used for every samples. The prepared cells were run in a Fluidigm Helios CyTOF MS machine and analyzed in Cytobank.

Multi-Color FACS of Aortic VICs

The 13 markers identified in the FACS analysis were tested for co-expression of progenitor and calcification markers by multi-color FACS. To determine surface markers associated with putative progenitor cell population, each of the 18 markers was tested for co-expression with the other, and co-expression with a marker for calcification (osteocalcin) and a marker for proliferation (Ki-67). The FACS protocol was carried out. We washed and strained cells from valve digestion and then blocked with Fc blocker prior to surface antibody (fluorochrome conjugated) staining (three antibody cocktail combinations). After staining with surface antibodies, we washed, fixed, and permeabilized the cells with permeabilization buffer containing 1% saponin (eBioscience) to prepare for intracellular marker staining. Staining with a cocktail of osteocalcin and Ki-67 antibodies was followed by stringent cell washes and final cell fixation prior to FACS reading. FACS data were acquired using BD FACS Aria (BD Biosciences) and analyzed using FACS Diva software.

Fluorescence-Activated Cell Sorting (FACS of CD44^{high}D29⁺CD59⁺CD73⁺CD45^{low} Population

To isolate the CD44^{high}CD29⁺CD59⁺Ki67⁺OC⁺D45^{low} disease driver cells by flow cytometry, confluent early passage (P1-P4) heterogeneous VICs from at least one 150 cm2 flasks were detached and resuspended in FACS buffer consisting of DMEM, 1% penicillin and streptomycin, 3% FBS and 1 mM HEPES. After centrifugation at 300 g for 10 min, cell pellets were resuspended in an antibody cocktail solution with 4 μL APC-conjugated CD44, 3 μL of AF488-conjugated CD29, APC-conjugated CD59 and PE-conjugated CD73 and 1 μL of PE-conjugated CD45 in 200 μL FACS buffer in a microtube. All stock antibody solutions were at 0.5 □g/□L. We kept the sample(s) in the dark and let it incubate while being swirled and mixed in room temperature for 40 min. We added another 1 mL of FACS buffer before a spin-down at 500 g for 5 minutes. After discarding the supernatant, we repeated the washing step with FACS buffer. We resuspended the final pellet in 400 μL of FACS buffer. Two 15 mL receiving tubes were prepared, each with 4 mL of FACS sorting solution, containing DMEM, 1% penicillin and streptomycin, 5% FBS, and 10 mM HEPES. Cells matching CD44^{high}CD29⁺CD59⁺CD73⁺CD45^{low} were sorted out in one tube and the remaining cells in the other tube using the BD FACS Aria. Sorting efficiency was between 96-99% in purity mode.

Laser Microdissection of Calcified Aortic Valve Tissues with IHC Cross-Validation

We dissected calcified and non-calcified regions using two strategies: Manual dissection with a scalpel under the microscope (one donor leaflets), and laser microdissection (LMD) with the MMI CellCut™ device (two donor leaflets). LMD precision was enhanced by flash staining sections with hematoxylin-eosin cocktail (MMI) (showing cell nuclei) to verify that VICs are contained within the LMD samples extract the cells from the specified areas in valves. The isolated cells (via LMD samples) were then processed for mass spectrometry (further below).

Sample Preparation for Proteomic Analysis

Protein Isolation and Peptide Digestion from Calcified Aortic Valve Tissue

The cell pellet collected was sonicated on ice at 4° C. (cold room) using a Branson Sonifire 450 (Branson) 4× for 15 seconds at constant duty cycle and power output of 2 in 30 second intervals. Further peptide preparation was conducted using the iST 96× Kit (PreOmics GmbH), strictly following the manufacturer's protocol, with samples normalized to 15 μg protein input, measured with a BCA protein quantitation assay (Thermo Fisher Scientific) using Nanodrop2000 Spectrophotometer (Thermo Fisher Scientific).

Protein Isolation and Peptide Digestion for Time Course Profiling of Differentiated MSCs and VICs

The cell lysates in RIPA buffer (see In vitro modeling of aortic valve calcification) were thawed and slowly passed through a pre-chilled gauge 27 needle using a 1 mL-syringe over ice 20× to facilitate and complete cell lysis. Each of the resulting homogenate-lysates was first processed by sonicating on ice as described above. The protein disk precipitate from the sonicated homogenate was extracted using a 2:1 chloroform:methanol solution through vigorous vortex mixing for 30 seconds followed by high speed centrifugation at 18,000 g for 30 minutes at 4° C. Upper and lower liquid phases were discarded and each protein disk was solubilized in lysis buffer of the iST 96× Kit using 50 μg protein input.

Mass Spectrometry

For data-dependent acquisition (DDA, unbiased peptide sampling), we analyzed peptides using the Orbitrap Fusion Lumos Tribrid mass spectrometer (Thermo Fisher Scientific), fronted with an Easy-Spray ion source and coupled to an Easy-nLC1000 HPLC pump (Thermo Scientific). The peptides were separated using a dual column set-up: an Acclaim PepMap RSLC C18 trap column, 75 μm×20 mm; and an EASY-Spray LC heated (45° C.) column, 75 μm×250 mm (Thermo Fisher Scientific). The gradient flow rate was 300 nL/min from 5 to 21% solvent B (acetonitrile/0.1% formic acid) for 120 minutes, 21 to 30% Solvent B for 10 minutes, followed by five minutes of 95% solvent B. Solvent A is 0.1% formic acid. The instrument was set to 120 K resolution, and the top N precursor ions in 3 seconds cycle time (within a scan range of 375-1500 m/z) are subjected to collision induced dissociation (CID, collision energy 30%) for peptide sequencing (or MS/MS).

The MS/MS spectra were queried against the human UniProt database (downloaded on Aug. 1, 2014; number of sequences equals 88,944) using the HT-SEQUEST search algorithm, via the Proteome Discoverer (PD) Package (version 2.2, Thermo Fisher Scientific). The precursor mass tolerance was set to 20 ppm and the fragment mass tolerance was set to 0.5 Da. Methionine oxidation and n-terminal acetylation were set as variable modifications, and carbamidomethylation of cysteine was set as a fixed modification. Peptides were filtered based on a 1% FDR based on the reverse database (decoy) results (Elias J E & Gygi S P (2007) Nature Methods 4(3):207-214; Kall et al., (2008) Journal of Proteome Research 7(1):29-34). In order to quantify peptide precursors detected in the MS1 but not sequenced from sample to sample, we enabled the ‘Feature Mapper’ node. Chromatographic alignment was done with a maximum retention time (RT) shift of 10 minutes and a mass tolerance of 10 ppm. Feature linking and mapping settings were, RT tolerance minimum of 0 minutes, mass tolerance of 10 ppm and signal-to-noise minimum of five. Peptides assigned to a given protein group and not present in any other protein group were considered as unique. Consequently, each protein group is represented by a single master protein (PD Grouping Feature). Precursor peptide abundances were based on their chromatographic intensities and total peptide amount was used for normalization. We used unique and razor peptides per protein for quantification (Tyanova et al. (2016) Nature Protocols 11:2301). Proteins with two or more unique peptides were considered for further analysis. The proteins from each Proteome Discoverer exported dataset were normalized by the protein median intensity. The protein abundance trends (heat maps and principle component analysis) were analyzed using Qlucore Omics Explorer 3.4 (Qlucore AB, Lund, Sweden). Using Qlucore, we also looked for differentially regulated proteins between different regions of the valve.

Analysis Workflow of MSC and VICs Timecourse Proteomics

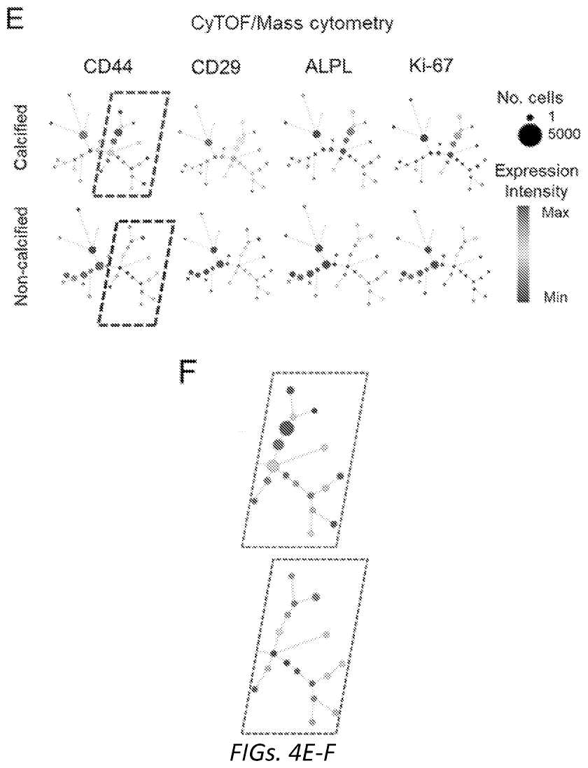

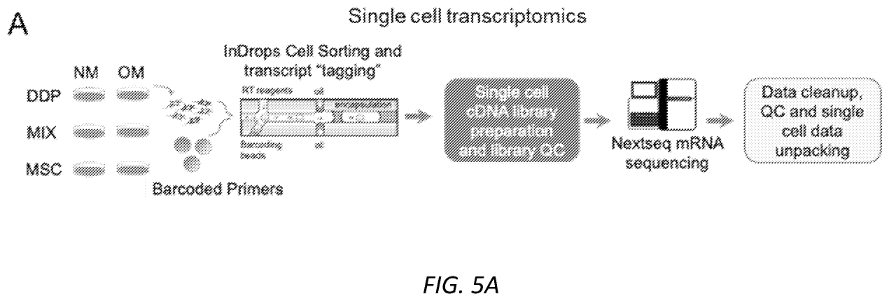

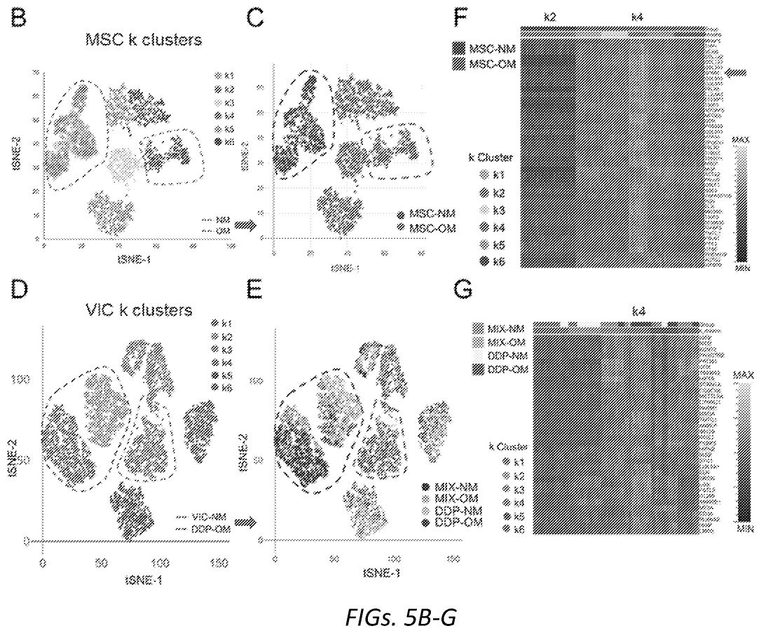

Based on our previous study (Schlotter et al., Circulation. 2018 Jul. 24; 138(4):377-393), we expected that for the VICs, in addition to the time and media condition, a third source of variation would be baseline donor-to-donor variability. Initial PCA analysis of the VIC data showed that samples plotted distances on the PCA plot axes were mainly driven by donor effects, thereby masking the underlying biology related to the differentiation time course. This donor-to-donor variability and effect was removed by factor elimination using a general linearized model in Qlucore OMICs explorer (Wichura, In The Coordinate-Free Approach to Linear Models. Cambridge University Press (Chapter 6):110-136 (2006)). In order to identify similarly expressed proteins in MSCs and VICs as a function of OM-induced differentiation, we performed a series of filtering steps (FIG. 5F). Specifically, for MSCs we filtered for proteins that were differentially expressed in OM vs. NM; and in parallel we filtered for those that whose abundances increased over time in OM. As a first step, protein abundances were filtered using rank regression across replicates over time (q-value <0.005). The analysis was conducted separately for the samples in OM and NM. To exclude proteins whose abundance changes are driven by cell culture, we omitted those that remained in the filtering steps for both OM and NM conditions, narrowing down the MSC global proteome from 3998 to 328 proteins (q-value <0.005). In parallel, we performed statistical filtering of differentially expressed proteins (DEP) of the original MSC global proteome by two-group comparison of NM vs. OM at 7, 14, and 21 days respectively (cut-off q-value <0.005). In order to prioritize filtering based on media condition and not the confound of time point, we added a factor elimination for time as described earlier, resulting in a reduction from 3998 to 956 DEP (increase and decrease, inclusive) between OM and NM (FIG. 5F).

From the global proteome, sample conditions (MSC-OM, MSC-NM, DDP-OM, DDP-NM, MIX-OM, and MIX-NM) were analyzed separately as 2 main comparisons initially: (1) MSC-OM vs. MSC-NM, (Krishnamurthy et al. (2017) Matrix biology: journal of the International Society for Matrix Biology 62:40-57) and (2) comparison of DDP-OM, DDP-NM, MIX-OM, and MIX-NM. In the MSC-OM vs MSC-NM differentially expressed proteins comparison, analysis was done two ways: a rank-regression across time points (0 weeks, 1 week, 2 weeks and 3 weeks after either OM or NM culture) and a two-group comparison between media conditions (OM vs. NM). The resulting differentially expressed proteins (DEP) as time progresses from 0 weeks to 3 weeks as filtered through rank regression were consolidated to and designated as “DEP MSC-OM”. The DEP MSC-OM that increases over time comprise of 86 proteins.

In the analysis workflow of DDP and MIX proteomics data, from the global proteome, sample conditions were analyzed, as above, in two ways: a rank-regression across time points and a two-group comparison between media conditions. To identify differences in the proteome of these VIC samples (FIG. 5H) Thus, we performed all filtration steps for DDP and MIX in parallel until consequent convergence of the data. The first rank regression filtering step resulted in a reduction from 4538 to 1002 proteins (q-value <0.05) for the VIC-MIX, while the VIC-DDP reduction was from 4444 to 747. The two-group comparison between NM vs. OM conditions filtered 679 DEP for MIX and 629 DEP for DDP VIC, (q-value <0.005). The overlap between the DEP lists from the two statistical filtering schemes, for each VIC condition, resulted in 330 DEP for MIX and 289 DEP for DDP. For an uncomplicated straightforward analysis, we further limited the 330 MIX DEP and 289 DDP DEP list by focusing on the set of proteins with an increasing-over-time trend (197 & 169 proteins), analogously to the DEP of MSCs.

Exploring these OM-vs.-NM- and time course-derived DEP lists further, may reveal proteins that may explain the phenotypical difference between DDP and MIX. By taking the overlap between the MIX-DEP (197) and the DDP-DEP (169), non-discriminating proteins (shared) can be eliminated from downstream analysis (FIG. 5F). There are 97 proteins shared between the MIX (197) and DDP (169), while 71 proteins are exclusive to the DDP (FIG. 5I). These 71 proteins are differentially expressed over time while being differentially expressed between NM and OM conditions, among the DDP.

In Vitro Modeling of Aortic Valve Calcification

For in vitro experiments, either heterogenous VICs or ones isolated by flow cytometry were expanded in cell culture to conduct proteomics as well as single-cell transcriptomics in a time course setup. In addition to VICs, human adipose-derived mesenchymal stem cells (ATCC® PCS-500-011™) were used as a positive control in osteogenic media (OM) and as a negative control in growth media (GM). Cells were lysed at time points of 0, 7, 14 and 21 days in OM and GM in either 10% RIPA buffer (CST) in HPLC H₂O containing 1% protease inhibitor cocktail (Sigma) by vigorously scraping the cells with polyethylene cell scrapers to collect them or immediately frozen and stored in −80° C. until further processing. Additionally, the media at the according time points was collected for future Enzyme-linked Immunosorbent Assay (ELISA).

Functional In Vitro Assays (MSCs and VICs)

Osteogenic, chondrogenic, and adipogenic differentiation assays were conducted for samples of protein, RNA, and media collected at 0, 1, 2, and 3 weeks after changing media from growth media to specialized media. Alizarin Red. alizarin red (AR) 2% (Thermo Fisher Scientific), Alcian blue (AB) 1%, and oil red O (ORO) (Abcam) were used for osteogenic, chondrogenic, and adipogenic assays respectively. Cells plated in 48 or 96 well plates were washed with PBS and fixed for 15 minutes at room temperature with 10% formalin for AR and AB and 4% PFA for ORO. After washing with Milli-Q water, stains were added. For ORO, another washing step with propylene glycol (85%) with 2 minutes incubation was conducted. The incubation times were 20 minutes (AR), 1 hour (AB), and 15 minutes in room temperature (ORO). Washes with Milli-Q water followed for the AR (3×) and AB (4×) stains. For ORO, the wash steps were as follows: propylene glycol for 5 minutes, followed by 5× washing with Milli-Q water, 2 minutes incubation with hematoxylin, and a final 5× wash with Milli-Q water was conducted to finish the staining process. Pictures were taken using an iPhone7 (Apple) and the amount of dye released was quantified by spectrophotometry at 550 nm.

Computational Analysis of Proteomic Data and Network Analysis

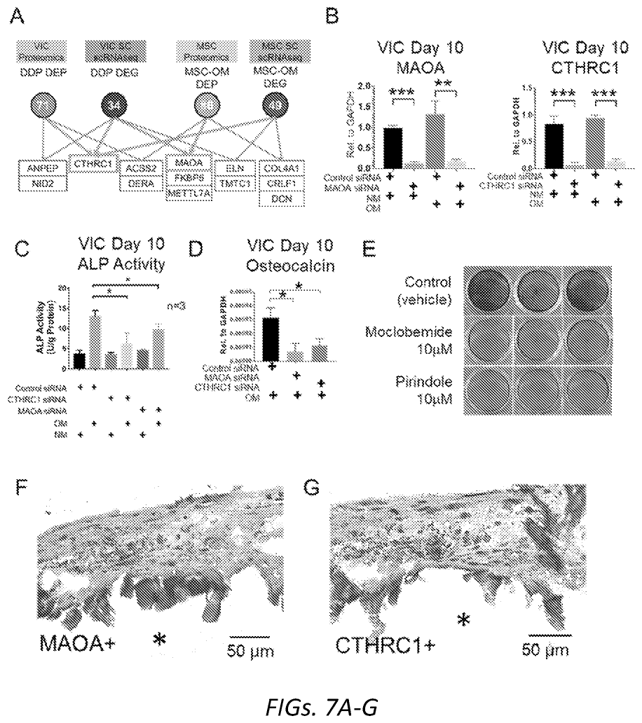

Using the multi-group comparisons with FDR<0.5, we statistically filtered the proteomic data between the different groups. Using Qlucore, we conducted network-based analysis including pathways enrichment to find significant disease associations and potential therapeutic targets among ‘hub’ nodes using the proprietary computational algorithms of MetaCore™ (Clarivate Analytics/Thompson Reuters). In addition, we built subnetworks of the filtered proteomics data and calcification and DDP surface markers using a global map of human protein-protein interactions (PPI). The human PPI network consists of curated physical PPIs with experimental support, including binary interactions, protein complexes, enzyme-coupled reactions, signaling interactions, kinase-substrate pairs, regulatory interactions and manually curated interactions from literature, the details of which were described previously (Menche J, et al. (2015) Science 347(6224):1257601) The subnetworks depicted in FIG. 7 were constructed by mapping the filtered proteomics data and calcification and DDP surface markers, along with their direct interactors (first neighbors), onto the PPI network using the Networx library v1.9 in Python v2.7.10. Shared proteins between subnetworks were identified to determine the overlap between subnetworks. The statistical significance of the overlap between subnetworks was calculated using two-tailed Fisher's exact test. Networks were visualized using Gephi v 0.9.2.

Single Cell Transcriptomics Methods

Single cell isolation and cDNA library preparation vis I(7)nDrops—Cells were detached from culture by adding 1.0 mL/10-cm² area of Accutase (BD Biosciences) and incubating at room temperature for 3 minutes. VICs normal media (5% FBS+DMEM) was added to stop the enzymatic digestion. Single cell dispersion and preparation was achieved by gently pipetting up and down 20× and passing the cells through 70 um cell strainer (Corning). Cells were then immediately sorted and processed using in-droplet barcoding according to a previous report (Klein A M, et al. (2015) Cell 161(5):1187-1201).

cDNA-library preparation and sequencing—The cDNA library was prepared using the CEL-Seq/MARS protocol with quality control analysis done with Agilent Bioanalyzer. Superscript III Reverse Transcriptase (Invitrogen)/cell lysis mix was encapsulated a priori in the hydrogel droplets together with primer beads. We sequenced with an Illumina NextSeq sequencer using a multi-read approach. The first read acquired the barcodes of the samples and the universal molecular identifiers (UMI) sequences, while the second read mapped the results to a reference transcriptome. Sequencing was done at the Dana-Farber Cancer Institute Molecular Biology Core Facilities (MBCF).

Single Cell Analysis Methods

Raw data processing to normalized UMI-filtered counts—Post sequencing, raw FASTQ data files underwent bioinformatics processing via bcbio/bcbio nextgen-seq, a python toolkit (github.com/bcbio/bcbio-nextgen) and algorithm pipeline. Briefly, Nextseq reads were assigned to each cell by matching the cellular barcodes followed by the UMI extraction for each read (Svensson et al. (2016) Power Analysis of Single Cell RNA-Sequencing Experiments. bioRxiv. dx.doi.org/10.1101/073692 and Nature Methods volume 14, pages 381-387(2017)). The reads were then aligned to the hg38 transcriptome (augmented with the transgene) using RapMap (Ciceri et al. (2016) Calcified Tissue International 99(5):472-480) (doi.org/10.1093/bioinformatics/btw277) and counts of reads per transcript per unique UMI were generated for each cell. A multi-step quality control aimed to filter out any noise in the raw data was performed. As a first step, only cells with more than 1,000 reads were kept using the bcbio filtering pipeline. The distributions of reads per cell, UMIs per cell, genes per cell, and mitochondrial ratio per cell were used to further define cutoffs, thus identifying cells with high quality RNA. Additional QC metrics applied include UMIs vs. genes detected, UMIs vs. read counts, and a novelty score. The novelty score method helps filter out cells that have less genes detected per count than other cells by looking at the genes per UMI. The final data was exported in form of a cell by gene matrix for each sample. Data preparation was conducted at the Harvard T. Chan Bioinformatics core facility.

SeqGeq analysis_—The data which had gone through QC was analyzed using SeqGeq software with SeqGeq plugins installed from FlowJo Exchange (exchange.flowjo.com). As a first step, each necessary sample-condition cell population for the analysis was concatenated as one file. From this concatenated file, a cell dispersion plot was used to exclude any doublets missing from the bcbio QC doublet exclusion algorithm. We found almost no doublets in our post-bcbio analysis. A gene dispersion plot was used to gate-out highly expressed and very lowly expressed genes (≤10 genes per cell), globally. Filtering out these “outlier” genes was necessary since very high expression among all cells may be indicative of housekeeping features, or mitochondrial genes, while very lowly expressed genes (expressed in ≤10 cells) may contribute noise to downstream clustering and are most likely low quality reads. Gene data points falling within the 10-1,500×10-1,500 gate window were labeled “outlier-filtered genes”, which are free of the outlier genes (highly expressed ≥1,500 counts/cell or lowly expressed ≤10 counts/cell. By switching to gene view, using only the outlier-filtered gene set, we plotted genes in a scatterplot bound by axes of “cells expressing-” vs. “gene dispersion” features and gated for the highly dispersed genes (HDGs) that vary expression considerably among cells. This gate excluded the area containing plot coordinates coinciding with the “control” spiked RNA species. These external RNA controls consortium (ERCC) RNA InDrops spiked sequences served as non-varying control RNA species within our samples and confirmed the appropriate boundaries of the HDGs gating strategy as well as testing for batch variability among samples. The HDGs gene set was used as basis for computing for the principal component analysis (PCA) visualization of the single cells.

High dimensionality reduction was initially done by PCA using the highly dispersed/varying gene expression of the HDG gene set. The resulting top 25 PCA components describe the gene parameters that explain the majority of the gene expression variance among cells. These may be due to the sample condition, cell type of the samples or intrinsic cell population heterogeneity within each sample-condition. Subsequently, the top 15 PCA components were used to conduct non-linear dimensional reduction through t-Distributed Stochastic Neighbor Embedding (t-SNE). The resulting t-SNE plots clustered cells based on global gene variance. In conjunction with multigraph color mapping, the phenotype of “islands” of cells in t-SNE space becomes clear. Using stepwise filtering of highly variant genes from QC to PCA to t-SNE mapping was particularly effective in clarifying cell cluster “calling”.

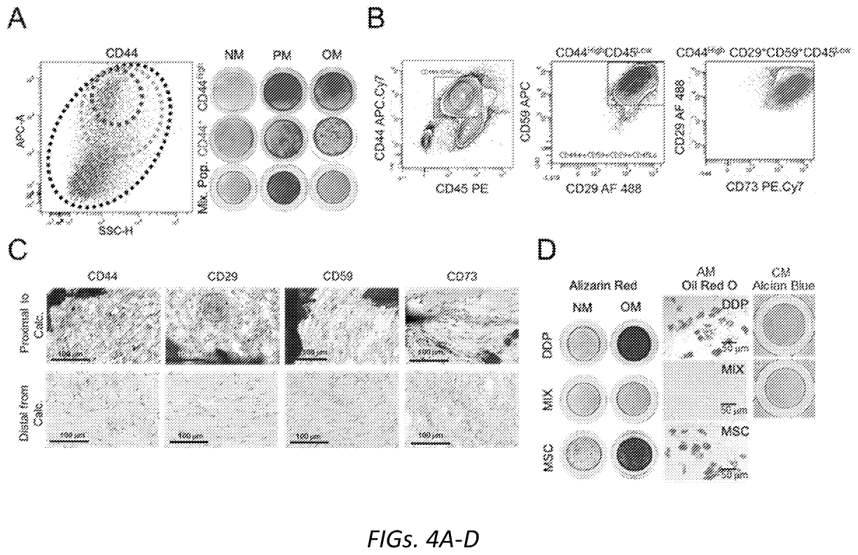

Differentially expressed genes (DEG) analysis of single cell transcriptomics—After initially analyzing all 6 samples (MSC-OM, MSC-NM, PUR-OM, PUR-NM, MIX-OM and MIX-NM) as one concatenated data frame, we found that MSC samples tend to cluster distinctly from the VIC samples, suggesting that few gene expression patterns are shared between VICs and MSCs, despite the polarizing effect of either the osteogenic media (OM) or normal media (NM). To derive potential shared biology between the OM-modeled VICs and the prototypical vascular calcification model (OM) MSCs, we analyzed MSCs and VICs as separate concatenated files. The resulting 2 separate differentially expressed gene sets (MSC- and VICs-specific) were then analyzed as 2 separate OMICs summaries. Therefore, we analyzed the MSC dataset first separately. We processed the MSC dataset using SeqGeq as above. This time we used HDGs only from the MSC dataset. The resulting PCA processing revealed that only the top 6 principal components (PC) were needed to create a tSNE plot, because the top 6 PC explains most of the variability of the MSC dataset. Then, k-means clustering was done using these top 6 PC components with the intent of separating the cells into 6 k-means clusters. We decided to limit ourselves with 6 clusters because it only takes the 6 PCA components to explain most of the MSC variability. Intuitively, we interpreted this such that the MSC dataset may be minimally separated into 6 “communities” if it takes 6 PCA components to explain most of the variability. To recreate a more separated tSNE plot, we simply performed a redundant tSNE plotting this time using the 6 PCA components plus the k-means components (class/cluster assignments based on k-means clustering) resulting in a secondary final tSNE plot (FIG. 4A-B).

We processed the VICs dataset similarly and found that there are also only 6 top PCA components that can explain most of the variability in the VICs dataset. Because of this, we processed the data in the same fashion as the MSCs to get a final tSNE plot showing good separation of VICs k-means clusters (FIG. 4E-F).

We first filtered for increased differentially expressed genes (DEGs) among the MSC-OM samples by volcano plots of fold change vs. −log (10) q-value (false discovery rate, FDR). There are 43 DEGs in the MSC-OM with an increased fold change of ≥1.5 at q-value ≤0.05 relative to the MSC-NM (FIG. 4D). Upon comparison between each MSC-OM cluster however, we found that there were no set of DEGs that would uniquely mark one MSC-OM k-means cluster over another, suggesting that MSCs being a cell line is much less heterogenous than we initially expected. However, the seeming heterogeneity plotted after tSNE and k-cluster differentiation, is explained by the fact that the clustering of MSC-OM cells depended mainly on the similarity of transcript counts (intensity of expression) for each of these 43 OM-DEGs within MSC-OM cells belonging to the same k-means clusters. Still, by interrogating the expression of a known osteoblast associated marker, SPARC (11) (FIGS. 4C&D), we identified MSC k4 cluster as the most “pro-calcifying” cluster of MSC cells. By filtering only, the DEGs in k4 MSC over k1 (MSC-NM) at FC≥1.5 q≤0.05 we identified 50 DEGs that may be applicable as calcification targets for VICs as well.

We analyzed the VICs dataset similarly and identified that k4 k-means cluster is the most interesting cluster to explore (see Examples below). We therefore compared the k4 cluster against the combined k1 and k2 clusters which contain mostly VIC-NM cells (DDP and MIX) to identify enriched DEGs by plotting volcano plots and gating for genes that have a fold change ≥1.5 and −value ≤0.05

In Vitro Silencing

Silencing experiment was performed in VICs cells from human AOVs (n=3). Cells were plated in 48 well plates and were treated with siControl, siMAOA and siCTHRC1. Lipofectamine RNAiMAX Transfection Reagent (Thermo Fisher) was used as per manufacturer's protocol for this experiment. Before transfection, cells were treated with normal medial and osteogenic media. Silencing experiments were performed using each siRNA in each media condition. Cells were harvested in two different time points for RNA extraction (3 days and 10 days). For TNAP activity, cells were harvested in 10 days. Transfection was repeated every 3 days.

RNA Purification and cDNA Synthesis

RNA isolation was accomplished using the Illustra™ RNAspin mini kit (GE Healthcare, Cat #25-0500-72). RNA Lyse solution from the Illustra RNAspin Mini Kit (GE Life Science) mixed with 1% 2-mercaptoethanol (Sigma) was added to the adherent cells. Each lysate was frozen at −30° C. until further purification. RNA purification was done as per manufacturer's protocol with the GE Healthcare Illustra™ RNAspin Mini Isolation Kit (lot 1711/001). The final purified RNA was eluted in 16 μL RNase-free H2O for a high concentration of RNA. The concentration of each sample was measured using a NanoDrop Microvolume Spectrophotometer 2009 (ThermoFisher). To normalize the amount of RNA used between samples, we calculated the volume needed for each sample to have 500 ng of RNA. Complementary DNA (cDNA) was made using the qScript cDNA Synthesis Kit (Quantabio). The volume of RNA was added to a strip tube and diluted with nuclease free water to a total volume of 15 μL. We added 4 μL of reaction mix and 1 μL of reverse transcriptase (RT) per strip tube. After centrifugation, the prepared strip tube plate was processed for cDNA synthesis in Biosystems 2720 Thermal Cycler. The final samples were stored at 4° C.

Pre-Amplification

Pre-Amplification of cDNA samples was performed using the Perfecta PreAmp SuperMix (5×) kit (Quantabio). An assay primers pool using 2 μl of each primer and additional TioEo, buffer (10 mM tris-HCl (pH 8.0), 0.1 ml EDTA) to reach 200 μl total volume was made. The pre-amplification reaction mixture for TaqMan assays with a total volume of 20 ul was mixed, using 4 μl of PerfeCTa PreAmpl SuperMix (5×), 2.5 μl of TaqMan Assay Pool, 5 μl of cDNA and 8.5 μl of nuclease free water. Each sample was incubated in a thermal cycle, following these steps: Initial denaturation (95° C., 2 minutes), PreAmpl cycling (14 cycles of 95° C., 10 seconds; 60° C., 3 minutes) and hold (4° C.). Concurrent samples were than used for qPCR.

qPCR (Quantitative Polymerase Chain Reaction)

For qPCR analysis, we used a 384 well plate with 2 μl of PreAmp cDNA and 8 μl of primer cocktail per well. The primer cocktail was made with 5 μl of TaqMan Fast Universal PCR Master Mix (2×), 2.75 μl of nuclease free water (QuantaBio) and 0.25 μl of respective primer. PCR was performed using 79020HT Fast Real-Time PCR Systems. Data analysis was made using Prism GraphPad 8.

Tissue Non-Specific Alkaline Phosphatase Activity (TNAP) Assay

TNAP assay was done using the Alkaline Phosphatase Activity Colorimetric Assay Kit (BioVision, Inc, Cat #K412-500) as per manufacturer's recommended protocol. Each of the sample lysates were taken from a well of a 48-well plate in triplicates. Measurements were calibrated against a standard curve and normalized with protein amount.

Immunohistochemistry of Calcified Valves

Tissue sections from CAVD patient's aortic valve leaflets (n=10) with the evident three-layer structure were immunostained for putative fibroblast and progenitor markers: CD34, CD44, CD45, CD73, CD90, CD105, CD133, CD146, NANOG, C-Kit, osteocalcin, and vimentin. For staining MAOA and CTHRC, a separate set of CAVD patient sourced leaflets (n=3) were used. Standard immunostaining protocols described herein were performed with primary antibody incubation done for 90 minutes, followed by conventional secondary antibody biotin-streptavidin peroxidase-based detection. All sections were counterstained with Harris-hematoxylin (Thermo Fisher) before imaging scanning (Omnyx VL4 scanner (GE Healthcare)) and image processing (either with sci-kit python package or Nikon Elements software version 3.20).

Cell Isolation and Culture

Valvular interstitial cells (VICs) were derived from aortic valve leaflets (n=12) obtained during aortic valve replacement surgeries in CAVD patients. Each leaflet was dissected for histological analysis and VICs isolation. VICs were isolated by collagenase IA-S digestion (1 mg/mL, Sigma). Cells were either immediately processed for flow cytometry or sorting, or cultured in DMEM with 10% FBS, then expanded further for downstream assays. Mesenchymal stem cells (MSCs) were obtained from ATCC (PCS-500-011) and expanded. In the fourth passage, cells were set up as either undifferentiated (normal media, NM) or differentiated (osteogenic media, OM; adipogenic media, AM; or chondrogenic media, CM) cells to use as reference sample conditions for succeeding experiments.

High Throughput Screening Flow Cytometry (HTS-FC) and FACS Sorting

VICs of calcified aortic valves and MSCs (after differentiating in NM, OM, and AM conditions for two weeks) underwent high throughput screening flow cytometry (HTS-FC) as per manufacturer's instructions. Immediately after digestion of the tissue, cells for each valve donor were cryopreserved until enough valves have been collected (n=8) and were then pooled to perform HTS-FC. Similarly, cultured MSCs (after NM, OM, and AM conditions for two weeks) were processed for HTS-FC using a 242 unique single antibody surface marker-per-well kit (BD Bioscience Lyoplate Human Cell Surface Marker Screening Panel) to profile surface marker expression. HTS-FC was read using BD FACS Canto II, and data was analyzed using Cytobank and FlowJo 10.7 software. Network analysis was done using MetaCore (Clarivate) and evaluated using Cytoscape and Gephi. Upon identifying surface markers for the disease driver population (DDP) of VICs, cells were FACS sorted for in vitro expansion and further functional assessment.

Single-Cell mRNA Sequencing

FACS sorted DDP-VIC, and non-sorted MIX-VIC were obtained from an enzymatically dispersed calcified aortic valve leaflets and in vitro propagated up to the fourth passage when both VIC types were cultured in NM or OM (stimulation culture). Osteogenic differentiation was assessed by alizarin red staining. Similarly, the fourth passage of MSC was included for comparison. Cells were harvested at day 14 of stimulation culture and processed for a single cell (sc) capture and sc-mRNA sequencing using InDrops. A limit of 2000 cells per sample condition and at least 1000 gene-reads per cell were set. scRNA-seq data was analyzed using the bcbio and SeqGeq software.

Proteomics Profiling In Vitro

MSCs (n=3 replicates), DDP-VICs, and MIX-VICs (n=3 donors) were cultured in NM and OM up to 21 days. Samples were harvested for several time points: Day 0, 7, 14, and 21, then processed and analyzed for proteomics, as shown previously. (Krishnamurthy V K, et al. (2017) Matrix biology: journal of the International Society for Matrix Biology 62:40-57) Data analysis was carried out using Proteome Discoverer (PD) Package (version 2.2, Thermo Fisher Scientific) and Qlucore Omics Explorer 3.4 (Qlucore AB, Lund, Sweden).

Functional In Vitro Assays

VIC cells isolated from CAVD valves were cultured up to the third passage before functional assay to validate identified potential calcification targets MAOA and CTHRC. Cells were treated with non-specific siRNA and MAOA or CTHRC siRNA up to Day 10. Intracellular alkaline phosphatase assay (BioVision, CA) and qPCR (MAOA, CTHRC, Osteocalcin, and Vimentin) were performed on replicate sample condition wells. To further prove the functional effect of MAOA inhibition, small molecule inhibitors moclobemide and bifemelane were used.

### Example 1. CD44 and Top Expressing Cell Surface Markers are Common Between MSCs and VICs

We hypothesized that a progenitor-like subpopulation among heterogeneous VICs might drive calcification in human CAVD. To explore this notion, we expanded mesenchymal stem cells (MSCs) (commercially purchased cell line from ATCC) as an experimental reference control. VICs harvested from eight additional donors were pooled and immediately processed for high-throughput screening—flow cytometric (HTS-FC) profiling of a library comprising 242 surface markers (FIG. 1A). This comparison determines the shared expression of markers of diseased VICs and undifferentiated MSC (MSC-NM), MSC differentiated towards osteogenic (MSC-OM), and adipogenic (MSC-AM) phenotypes (FIG. 1A).

The percent positive staining for each surface marker in the pooled VICs and the MSC-differentiated conditions was percentile ranked. We then gauged the percent positive cut-off based on previous reports of inclusion/exclusion of known MSC surface markers (35, 36). We found that at 85% cut-off, 16 surface markers consistently scored >85% percent positive in all three MSC conditions (NM, OM, and AM) and VICs, namely: CD13, CD29, CD44, CD47, CD49e, CD55, CD58, CD59, CD63, CD73, CD147, CD151, CD164, beta2-microtubulin, HLA A-B-C, and HLA-B2. In contrast, the percentage of CD38 and CD45 positive fractions, prototypical hematopoietic markers, were variable across the four samples.

To further filter and prioritize these 16 surface markers, we took a network approach to determine to what extent these proteins are associated with calcification-related molecules. Beta2-microtubulin, HLA A-B-C, and HLA-B2 are excluded in the analysis, as they are known to be expressed in all nucleated cells (37). The flow cytometry (FC) profiles rendered in a circular heatmap (FIG. 1B). By mapping the remaining 13 surface proteins onto a curated protein-protein interaction (PPI) network, we constructed a directed interactome network (FIG. 1B, inner panel). To anchor the network to calcification, we seeded four known calcification markers (osteocalcin, tissue-nonspecific alkaline phosphatase (ALPL), RUNX2, and osteopontin) as “target” nodes (blue nodes, FIG. 1B) (38-40). Each CD marker is a “source” node (red node, FIG. 1B). We used a network algorithm (MetaCore) that connects the source and target nodes through one “linking” node, at the most. The linking proteins generated included NANOG, a stem-cell proliferation marker (41). With these links, we ranked the 13 source nodes based on betweenness centrality and edge count, revealing CD44 as the central hub node (FIG. 1C).

### Example 2. CD44 Expression is Close to Calcification Regions in Aortic Valve Leaflets

To delineate potential cellular origins of VIC subpopulations, aortic valve leaflets from CAVD patients obtained from aortic valve replacement surgery and sectioned for immunohistochemistry (IHC; n=10). We assessed progenitor (12, 35, 42) and osteogenic (43) markers in longitudinal cross-sections of the leaflets: CD34, CD44, CD45, CD73, CD90, CD105, CD133, CD146, NANOG, C-Kit, osteocalcin, and vimentin. To characterize the layer-specific marker distributions, we used a Python script from the sci-kit repository to divide histology scans of entire stained leaflet cross-sections into the three anatomical layers (fibrosa, spongiosa, and ventricularis). The fibrosa layer is more calcification-prone than the ventricularis. (6) An interlayer comparison examining percent positive staining cells for the markers mentioned above was then conducted (representative valve, FIG. 1D). CD44 showed the highest percent positive staining among all cells in the fibrosa layer that are typically associated with the site of calcification. In contrast, its relative expression decreased in the ventricularis layer (FIG. 1E, p≤0.0001). This pattern of a decreased expression towards the non-calcified region is absent with any of the other tested markers (FIG. 1E).

### Example 3. CD44^{high} VICs are Functionally and Spatially Associated with Areas of Calcification

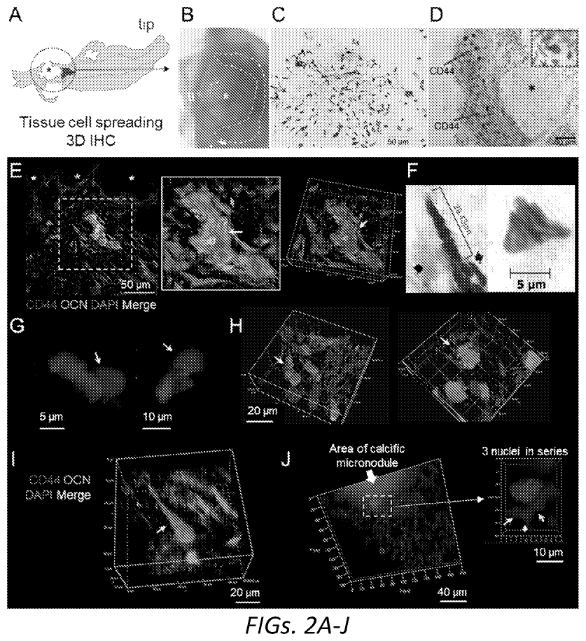

We then closely examined areas in which CD44 co-localizes with calcification by performing an immunohistochemistry-based tissue-spread technique (FIG. 2A-D, n=4 donors) that preserves the 3D tissue architecture, unlike routine histological sectioning-based methods (FIG. 1A). The region surrounding the calcification was dissected (FIG. 2A-B) and processed for tissue cell spread analysis (FIG. 2C) and CD44 immunostaining (FIG. 2D). Clusters of CD44-expressing cells appeared near microcalcification (representative image: FIG. 2D).

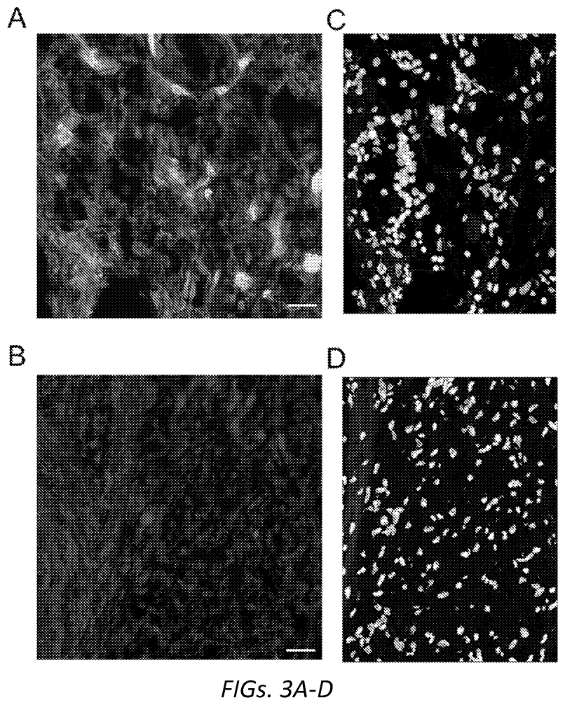

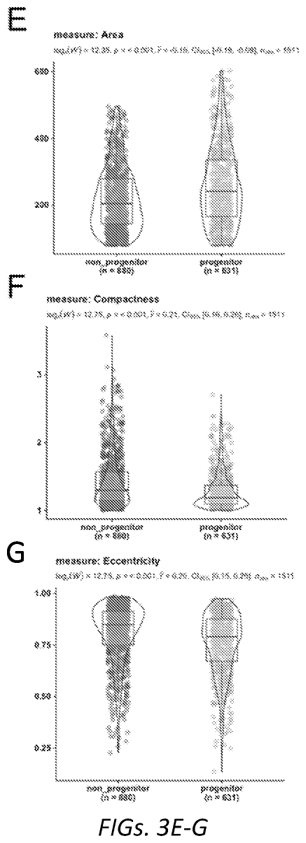

Z-stack confocal fluorescence microscopy demonstrated osteocalcin signals overlapping with CD44 staining in regions around microcalcifications (FIG. 2E). In aortic valve leaflets examined, we observed that a subgroup of cells having both CD44 and osteocalcin signals also harbor atypical nuclear morphology (FIG. 2E-J). These atypical nuclei may appear as stacked bell-shaped cups (FIG. 2F, left), reminiscent of metakaryotic progenitor cells found during human organogenesis (15) (FIG. 2F, right). Therefore, these CD44-positive VICs with cupped nuclei (FIG. 2G-J; yellow arrows) may be part of a pool of progenitor cells. We measured nuclei morphology between CD44 and CD29 double-positive cells near calcification areas (niche containing cells with atypical nuclear morphology) versus cells that are just CD44-positive CD29-negative far from calcification. Nuclear morphometric measures like area, compactness and eccentricity show a statistically significant difference between the groups of VICs (FIG. 3A-H). Confocal images were taken at 40× magnification and analyzed using Cell Profiler (FIG. 3F-G). Statistical testing done with Wilcox non-parametric rank sum.

To examine the calcification potential of CD44-positive VICs, we sorted them directly from enzymatically digested valve tissue using fluorescence-activated cell sorting (FACS). We sorted the CD44^{high} VICs within the general CD44-positive cell population (FIG. 4A). We sorted both CD44⁺ gated VICs (allophycocyanin (APC) mean intensity fluorescence (MIF)≥10³) and CD44^{high} VICs (APC MIF≥10⁴). The CD44^{high}, CD44-positive, and unsorted mixed population. VICs were cultured in normal media (NM) and two pro-calcifying media conditions—osteogenic media (OM) and high phosphate media (PM). OM is alkaline phosphatase (ALP)-dependent (44), while PM is an alkaline phosphatase-independent (45) mineralizing condition. Cells after 21 days of culture for calcification (Alizarin Red S). CD44^{high} and CD44-positive VICs calcified under both OM and PM conditions, but not in NM. CD44^{high} VICs calcified more readily than CD44-positive VICs (FIG. 4A). The unsorted mixed (MIX) VIC population only calcified in PM conditions. Furthermore, CD44 siRNA-treated VICs cultured in PM media reduced calcification to a level comparable to that of NM at three weeks (FIG. S3A).

### Example 4. Osteogenic CD44^{high} Cells Co-Express CD29, CD59, and CD73

Thus far, our findings demonstrate that higher calcification potential associates with CD44 expression in VICs. To further identify a fingerprint of this progenitor-like VIC population with higher calcification potential, we queried all 13 (source nodes) markers (FIG. 1B) for associations with the terms “mesenchymal stem cells,” “mesenchymal progenitor cells” and “calcification” using literature citation abstract retrieval. The eight most highly-cited markers for abstracts citing “mesenchymal stem cells” or “mesenchymal progenitor cells” were CD44, CD73, CD29, CD13, CD55, CD49e, CD147 and CD59 (ordered high-low). For markers overlapping with the term “calcification,” the ranking was CD29, CD44, CD13, CD63, CD73, CD59, CD55, and CD49e. The overlap of the two lists yielded CD44, CD29, CD13, CD73, CD55, CD59, and CD49e (a CD29 binding partner). We, therefore, assayed co-expression of these seven markers in CD44^{high} VICs using flow cytometry (n=3 donors). As a control for a non-mesenchymal-derived cell type, we monitored the hematopoietic marker CD45(32). We found that the CD44^{high} population was positive for CD29, CD59, and CD73 (FIG. 4B), and negative for CD13 and CD55. All tested VIC populations exhibited low staining for CD45 (FIG. 4B). IHC-based quantitative distance-from-calcification mapping of CD44, CD29, CD59, and osteocalcin positive cells showed a higher expression of these markers in closer proximity to calcification nodules. We thus defined our novel progenitor-like VIC population with high calcification potential as CD44^{high}CD29⁺CD59⁺CD73⁺CD45^{low}.

Aortic leaflet cryosections from three donors were then immunostained for CD44, CD29, CD59, and CD73, in addition to osteocalcin, Q-SMA (myofibroblast marker) and Ki67 (a nuclear marker of cellular proliferation (46)). VICs with proximity to calcification were immunoreactive to CD44, CD59, CD29, CD73 (FIG. 3C), osteocalcin, and Ki67 and colocalized with vimentin. Overall, these results demonstrated that VICs in CAVD contain a subpopulation of CD44-positive cells with high calcification potential and that this VIC subpopulation includes a subset of VICs that expresses the progenitor-associated phenotype CD44^{high}CD29⁺CD59⁺CD73⁺CD45^{low}.

### Example 5. The CD44^{high}CD29⁺CD59⁺CD73⁺CD45^{low} VIC Phenotype Mimics MSC-Like Properties In Vitro

We then tested the pluripotent ability of this VIC subpopulation, designated as the disease driver population (DDP-VICs), in contrast to the unsorted heterogeneous VICs designated as a mixed population (MIX-VICs). From additional CAVD leaflets (n=9 donors), we isolated, sorted, and cultured DDP VICs and MIX VICs and used MSCs as control. All MSCs were cultured in OM, adipogenic (AM), chondrogenic (CM), or NM (FIG. 4D) for three weeks to assess their osteogenic, adipogenic and chondrogenic potentials. Alizarin Red, Oil red O, and Alcian blue staining detected osteogenic and adipogenic differentiation respectively for both DDP and MSC populations, but not for the MIX. DDP in chondrogenic media showed positive Alcian blue staining but not MSCs in chondrogenic media (image not is shown). These findings suggest that CD44^{high}CD29⁺CD59⁺CD73⁺CD45^{low} DDP VICs have pluripotent progenitor cell-like properties with high osteogenic potential.

### Example 6. Mass Cytometry Verifies DDP-VICs Progenitor-Like Phenotype

We used mass cytometry (CyTOF) to evaluate phenotypic features of the DDP-VICs, by comparing calcified versus non-calcified regions of aortic valve leaflets. CyTOF offers more surface and intracellular markers screening simultaneously, with minimal spectral overlap compared to multi-color flow cytometry (47). We isolated VICs from the calcified and non-calcified portions of CAVD leaflets (n=4) and incubated the cells with 11 intracellular markers of CAVD recently described by us (6) (ALPL, α-SMA, GFAP, Ki-67, NANOG, osteocalcin, pS6, sortilin, TGF-β, VE-cadherin, vimentin) and eight surface antibodies (CD29, CD34, CD44, CD45, CD59, CD63, CD73, CD105) for subsequent CyTOF.

Spanning-tree progression analysis of density-normalized events (SPADE) visualization (48) demonstrates several cell clusters based on their expression profile of the measured markers, represented as nodes (FIG. 4E). Each node color represents the level of immunostaining (expression intensity: warm/red—high to cool/blue—low scale). We first identified a specific cell cluster containing DDP VICs by comparing the CD29, CD44 (FIG. 4E), CD59, and CD73 expressions in calcified and non-calcified regions of the leaflets. The cluster of VICs marked by trapezoidal boxes in FIG. 4E and shown enlarged in FIG. 4F demonstrates that calcified SPADEs contained more VICs with higher expression intensities (larger nodes with warmer colors) of CD29, CD44, ALPL and Ki67 (FIG. 4E), as well as CD59 and CD73 when compared to non-calcified SPADEs (smaller nodes with lower expression levels). NANOG, osteocalcin, pS6, sortilin, TGF-β, and CD34 also showed higher relative expression in the calcified samples, while α-SMA, GFAP, VE-cadherin, vimentin, CD45, CD63, and CD105 had no difference in staining in calcified vs. non-calcified SPADEs. These results suggest the predominance of a highly osteogenic (e.g., osteocalcin, sortilin, TGF-β), proliferative (e.g., NANOG, pS6), and progenitor-like (e.g., CD34) cell population in the calcified leaflet portion.

### Example 7. scRNA-Seq Identifies Highly Expressed Genes During the Osteogenic Transition of DDP VICs

We next examined the transcriptome at the single cell level by culturing DDP-VICs, MIX-VICs, and MSCs during osteogenic differentiation. Single cells from six conditions cultured for two weeks (to capture early mRNA changes before peak phenotype appearance in day 21) in NM and OM (MIX-NM, MIX-OM, DDP-NM, DDP-OM, MSC-NM, and MSC-OM) were isolated (FIG. 5A). InDrops scRNA-seq method was used. (49). The following are the number of cells per condition: MIX-NM (n=1813), MIX-OM (n=2000), DDP-NM (n=1814), DDP-OM (n=2000), MSC-NM (n=873) and MSC-OM (n=2000). The range of gene-reads per cell was from 1000 to 8192. Principal component analysis (PCA) and t-distributed stochastic neighbors embedding (tSNE) plots of the combined transcriptomics data revealed that at two weeks of differentiation, the MSC-NM and MSC-OM form distinct clusters that do not overlap with the other VIC clusters (MIX-NM, MIX-OM, DDP-NM, DDP-OM). This distinct separation between MSC and VIC samples (PCA and tSNE) suggests that their global transcriptome is substantially different and cannot be subjected to downstream co-clustering algorithms (e.g., k-means). We, therefore, analyzed the MSCs and VICs datasets separately to characterize VIC subpopulations (FIGS. 5B-G) further. The lack of MSC/VIC cluster overlap may imply substantial cell-type differences that drive global transcriptomic data divergence. However, there may still have some value in exploring MSC behavior during OM-stimulation, as this may allow us to capture some shared differentially expressed genes (DEGs) with the OM-stimulated DDP VICs or DEGs that are part of a shared pathway.

K-means clustering of concatenated MSC-NM and MSC-OM single-cell expression (intensity) data (6 clusters) as visualized by tSNE plots, showed that the MSC-OM population was more variable than that of MSC-NM since it separated into five subclusters (k1, k3, k4, k5, k6) compared to the single cluster (k2) depicting the entire MSC-NM population (FIG. 5B, C). On the other hand, variation distributed across all four VIC sample conditions was similar (FIG. 5D, E). We examined the relative gene expression profiles by calculating DEGs between MSC-NM and MSC-OM conditions without first considering the k-cluster groupings. The DEGs were organized by k-cluster for subsequent hierarchical clustering to filter for those with an increased fold change of ≥1.5 at q-value ≤0.05 relative to the MSC-NM (FIG. 5F). Of the five k-clusters that define the MSC-OM condition, k4 exhibits the highest expression for a number of these DEGs, including a known osteoblast-associated marker, SPARC (46) (FIG. 5D, arrow). As a further refinement to look for additional genes that differentiate the k4 cluster from the other MSC-OM k-clusters, we calculated the DEGs between k4 and k2 (MSC-NM) using a volcano plot (at FC≥1.5, q≤0.05), generating 49 DEGs.

Next, we interrogated the VICs-OM vs. VICs-NM for concatenated DDP-VIC and MIX-VIC samples. VICs-OM comprises of DDP-VIC and MIX-VIC samples in OM media (DDP-OM and MIX-OM), while VICs-NM is the combined DDP-VIC and MIX-VIC in NM media. Similarly, we analyzed the samples using PCA, tSNE, and k-means clustering. The VIC-OM clusters contain both DDP-OM and MIX-OM cells in varying numbers but were clustered together based on the similarity of overall gene expression. Comparing clusters of VIC-OM (k4, k5, and k6) vs. clusters of VIC-NM (k1 and k2) (fold change >1.5, q-value <0.05), we identified 18 increased DEGs (Table 1).

TABLE 1
Differentially expressed genes (DEG) of VIC-OM k clusters
over VIC-NM only k clusters. 18 DEG that were increased
by a fold change of >1.5 at q-value <0.05 in VIC-OM group
(k3-k6 inclusive together) as compared against VIC k-means
clusters that contain only VIC-NM cells (contains both
DDP-NM and MIX-NM).

| Gene name | Description |
|---|---|
| ANPEP | alanyl aminopeptidase, |
| | membrane(ANPEP) |
| CFH | complement factor H(CFH) |
| COL8A1 | collagen type VIII alpha 1 |
| | chain(COL8A1) |
| CTHRC1 | collagen triple helix repeat containing |
| | 1(CTHRC1) |
| ELN | elastin(ELN) |
| FKBP5 | FK506 binding protein 5(FKBP5) |
| HIF1A | hypoxia inducible factor 1 alpha |
| | subunit(HIF1A) |
| IGF2 | insulin like growth factor 2(IGF2) |
| KCNT2 | potassium sodium-activated channel |
| | subfamily T member 2(KCNT2) |
| METTL7A | methyltransferase like 7A(METTL7A) |
| NID2 | nidogen 2(NID2) |
| PHACTR2 | phosphatase and actin regulator |
| | 2(PHACTR2) |
| PROS1 | protein S (alpha)(PROS1) |
| PTGS1 | prostaglandin-endoperoxide synthase |
| | 1(PTGS1) |
| SCNN1A | sodium channel epithelial 1 alpha |
| | subunit(SCNN1A) |
| SGCG | sarcoglycan gamma(SGCG) |
| STC1 | stanniocalcin 1(STC1) |
| TGFBR2 | transforming growth factor beta |
| | receptor 2(TGFBR2) |

In this analysis, the osteoblast differentiation marker TGF-β receptor 2 (TGFBR2)(28) is increased among the VIC-OM. Subsequently, we filtered for DEGs from the clusters containing VIC-OM cells (k3-k6) compared to the clusters containing only VIC-NM (k1, k2). We included the k3 cluster among VIC-OM clusters despite containing cells from NM stimulation (FIG. 5E) because OM cultured cells predominate this cluster over NM-cultured cells. However, it is worth noting that the k4 cluster is predominantly DDP-OM (FIG. 5D-E). DDP-OM has a higher osteogenic potential than MIX-OM (FIG. 4D). The k4 cluster also has more cells with higher expression of ALPL and genes related to non-canonical WNT signaling important in aortic valve calcification (32) compared to k5 and k6 clusters, which are MIX-OM predominant (FIG. 5D-E). Using pathway enrichment, we identified the k4 cluster as having the most pathways related to stem cell differentiation and TGF-β signaling. Therefore, we were interested in k4 DEGs with ≥1.5-fold change over k1 and k2 combined clusters, which are almost entirely composed of MIX-NM and DDP-NM, yielding 34 DEGs (FIG. 5G, Table 2). Both MSC-OM k4 and DDP-OM k4 cluster-specific gene lists were enriched for biological processes and pathways as an in silico validation for genes responsible for driving calcification processes.

TABLE 2
Differentially expressed genes (DEG) in k cluster 4
(DDP-OM subcluster)/k cluster 2(DDP and MIX-NM)

| Genes | Description |
|---|---|
| ANKRD1 | ankyrin repeat domain 1 |
| ANPEP | alanyl aminopeptidase, |
| | membrane |
| CCDC68 | coiled-coil domain containing 68 |
| CD34 | CD34 molecule |
| CFH | complement factor H |
| COL8A1 | collagen type VIII alpha 1 chain |
| CTHRC1 | collagen triple helix repeat |
| | containing 1 |
| ELN | elastin |
| FKBP5 | FK506 binding protein 5 |
| FSTL3 | follistatin like 3 |
| HHIP | hedgehog interacting protein |
| HIF1A | hypoxia inducible factor 1 alpha |
| | subunit |
| HIPK2 | homeodomain interacting protein |
| | kinase 2 |
| IGF2 | insulin like growth factor 2 |
| IRS2 | insulin receptor substrate 2 |
| ITGA5 | integrin subunit alpha 5 |
| KCNT2 | potassium sodium-activated |
| | channel subfamily T membe |
| LMO3 | LIM domain only 3 |
| LOX | lysyl oxidase |
| MAOA | monoamine oxidase A |
| METTL7A | methyltransferase like 7A |
| MT2A | metallothionein 2A |
| NID2 | nidogen 2 |
| PARM1 | prostate androgen-regulated |
| | mucin-like protein 1 |
| PHACTR2 | phosphatase and actin regulator 2 |
| PLXNA2 | plexin A2 |
| PROS1 | protein S |
| RGS2 | regulator of G-protein signaling 2 |
| SCNN1A | sodium channel epithelial 1 alpha |
| | subunit |
| SGCG | sarcoglycan gamma |
| SRGN | serglycin |
| STC1 | stanniocalcin 1 |
| TGFBR2 | transforming growth factor beta |
| | receptor 2 |
| TMTC1 | transmembrane and |
| | tetratricopeptide repeat containin |

Fold change >1.5; q-value <0.05; n = 34 genes

### Example 8. Proteome Analysis of In Vitro Modeling of Aortic Valve Calcification

We next examined the changes to the cellular proteomes of DPP-VICs and MIX-VICs during osteogenic differentiation. Since large scale single-cell proteomics remains unfeasible, we performed subpopulation targeted proteomics on DDP-VICs and compared against the MIX-VICs isolated from the same donors (n=4). Alongside cultures of MSCs, these cell culture setups were grown in OM or NM conditions for 0, 7, 14, and 21 days (FIG. 6A). Alizarin Red calcification assays demonstrated that DDP VIC populations calcified by day 14 (resembling the calcification potential of MSCs), whereas all four MIX VICs donors exhibited calcification later at day 21 (FIG. 6B). Moreover, Alizarin Red staining was stronger in DDP-VICs compared to their MIX-VICs counterpart at the later time point.

We monitored the proteome during osteogenic differentiation to identify candidate drivers (proteins) of calcification in each cell population (MSCs, MIX-VICs, and DDP-VICs), and to determine whether these candidates are present in these cell populations. Proteomic analysis sequenced 3,998 proteins for the MSC-OM and MSC-NM combined, and 4,538 for the MIX-VICs and DDP-VICs combined, with 3607 (73%) proteins shared between the two cell types (FIG. 6C).

We first compared the baseline (t₀=day 0) proteome of the MSC-NM and MSC-OM using triplicate cell culture experiments (combined n=6 for day 0). These replicates demonstrate that the significant source of variation is between media conditions and time points, and not across sample replicates. When considering the combined condition proteome, the PCA depicted three significant findings: 1) the culture replicates show higher proximity within each cell population versus other sample conditions; 2) there is a time progression component that 3) also drives divergence between the MSC-NM and MSC-OM samples (FIG. 6D). Hierarchical cluster analysis provides an overview of the changing proteome across time points and media (FIG. 6E).

We then performed multiple statistical filtering steps to 1) reduce donor to donor variability, 2) model the impact of time in culture, 3) identify differentially expressed proteins over time in both the MSC and DDP-VIC populations, 4) model the effect of calcifying media treatment on these differentially expressed proteins. Full methodological details of our filtering approach are described herein and are depicted graphically in FIG. 6F, 6H.

The intersection of the two parallel filtering methods (FIG. 6F, 6H) resulted in 128 shared proteins. As a final step, we filtered for proteins enriched in OM over NM, resulting in 86 final proteins (FIG. 6G). To identify DDP-OM enriched proteins, we analyzed the DDP-VIC and MIX-VIC dataset separately and analogously to that of the MSC dataset; and incorporated an additional step that subtracted MIX-OM proteins from DDP-OM proteins (FIG. 6H) resulting in 71 proteins. (FIG. 6I).

### Example 9. CTHRC1 and MAOA are Identified by DDP-VIC Transcriptome and Proteome and Promote Calcification of VICs In Vitro

Combining scRNA-seq and in vitro proteomic approaches, we narrowed down our OM-mediated differential expression to 83 and 157 candidate molecules that may be drivers of osteogenic potential in DDP-VICs and MSCs, respectively. We then aimed to determine whether these proteins were shared between omics datasets and between these cell types. While this strategy gave rise to shared protein lists (FIG. 7A), none of the proteins were inclusive to all data sets. Proteins shared between multiple datasets, or cell types were ANPEP, NID2 CTHRC1, ACSS2, DERA, MAOA, FKBP5, METTL7A, ELN, TMTC1, COL4A1, CRFL1, and DCN. We specifically focused on two proteins: (1) CTHRC1 (collagen triple helix repeat-containing protein—the only shared molecule in the VIC-omics data (DEP and DEG lists) and the MSC-OM DEP list, and (2) MAOA (monoamine oxidase A)—present in the DDP DEG and MSC-OM DEP lists. Reports of MAOA, known for catecholamine degradation, show that high serotonin levels can cause oxidative stress in human heart valves (29). To determine whether these genes promote the calcification potential of VICs, we performed siRNA-mediated loss-of-function in VICs cultured for ten days in either OM or NM. The loss-of-function of MAOA or CTHRC1 (silencing efficiency was >90% for each gene, p≤0.0001, FIG. 7B) resulted in a decrease of ALPL activity and osteocalcin mRNA expression in VICs cultured in OM (n=3) (FIG. 7C, D). MAOA inhibitors moclobemide and pirindole demonstrated reduction of calcification (FIG. 7E) to support the above findings further. Currently, there are no CTHRC1 small molecule inhibitors available for parallel CTHRC1 testing. Immunostaining of CAVD tissue (n=3 donors) confirmed the enrichment of CTHRC1 and MAOA expression in regions near calcifications (FIG. 7E, 7F).

### Example 10. Calcification-Related Disease Networks Share Common Genes and Pathways with the Progenitor Markers Depicting DDP-VIC and Novel Targets Identified by OMICs

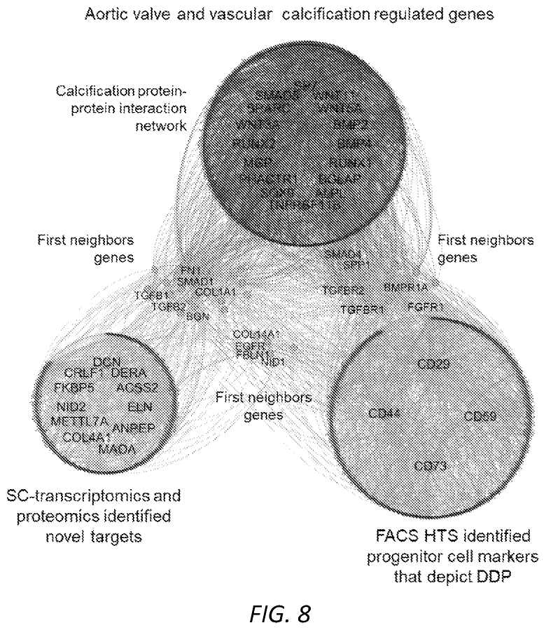

To determine the shared molecular constituents linking MSC-OM and VIC DDP-OM multi-omics targets with calcification and DDP-VIC surface markers, we mapped their respective gene sets onto the protein-protein interaction (PPI) network. Increasing evidence suggests that subnetworks that overlap in the PPI network may have similar functions, are co-expressed, or show comorbidity. (50) We, therefore, sought to demonstrate the network connections within these three datasets to identify the presence of shared protein interactions between the progenitor cell markers depicting DDP-VICs, the novel targets detected by proteomic profiling studies and DEG from the scRNA-seq study in association with calcification PPI network (FIG. 8). Overall, the subnetworks comprised of the genes enriched in MSC-OM and VIC DDP-OM omics data and DDP-VIC surface markers had a significant overlap in the calcification PPI network comprising calcification factors previously found to be associated with aortic valve and vascular calcification. This new network shared 27 genes with commonly associated calcification markers (Fisher's exact test, two-sided p-value=8.45e-12) and seven genes with DDP surface markers (Fisher's exact test, two-sided p-value=1,80e-04), suggesting common pathways connecting the three datasets. These common pathways that emerged from datasets derived from VIC profiling may be parts of a larger underlying mechanism of CAVD and agree with currently held concepts. (6)

### List of Abbreviations

ALPL tissue-nonspecific alkaline phosphatase
AM adipogenic media
APC allophycocyanin
aVICs activated myofibroblasts
CAVD calcific aortic valve disease
CD clusters of differentiation
CM chondrogenic media
CyTOF MS cytometry time-of-flight mass spectrometer
DDP disease driver population
DEG differentially expressed genes
ECM extracellular matrix
EVs extracellular vesicles
eVICs endothelial-derived VICs
FACS fluorescence-activated cell sorting
GM growth media
CAVD calcific aortic valve disease
HTS-FC high throughput screening-flow cytometry/flow cytometric
ICC immunocytochemistry
LMD laser-micro dissection
MMD manual-macro dissection
MIF mean intensity fluorescence
MIX unsorted mixed population VICs
miRNA microRNA
MMI hematoxylin-eosin cocktail
MS mass spectrometry
MSCs mesenchymal stem cells
OM osteogenic media
PCA principal component analysis
PM high inorganic phosphate media
PPI protein-protein interaction
PRM parallel reaction monitoring
PUR disease driver population or pure population VICs
ROI regions of interest
SAVR surgical aortic valve replacement
scRNA-seq single cell transcriptomic analysis
SMCs smooth muscle cells
SPADE spanning-tree progression analysis of density-normalized events
TAVR transcatheter aortic valve replacement
TGFBR2 Transforming growth factor-beta receptor 2
tSNE t-distributed stochastic neighbors embedding
VECs valvular endothelial cells
VICs valvular interstitial cells
XINA multiplexed isobaric mass tagged-based kinetics data for network analysis

### REFERENCES

1. Stone J H J, Willis M S, eds. (Aikawa E, Schoen F J.) (2014) Cellular and Molecular Pathobiology of Cardiovascular Disease (Calcific and degenerative heart valve disease). Elsevier: London, UK:161-168.
2. Coffey S, Cairns B J, & Jung B (2016) The modern epidemiology of heart valve disease. Heart 102:75-85.
3. Yutzey K E, et al. (2014) Calcific aortic valve disease: a consensus summary from the Alliance of Investigators on Calcific Aortic Valve Disease. Arteriosclerosis, thrombosis, and vascular biology 34(11):2387-2393.
4. Osnabrugge R L, et al. (2013) Aortic stenosis in the elderly: disease prevalence and number of candidates for transcatheter aortic valve replacement: a meta-analysis and modeling study. Journal of the American College of Cardiology 62(11):1002-1012.
5. Jung B, et al. (2003) A prospective survey of patients with valvular heart disease in Europe: The Euro Heart Survey on Valvular Heart Disease. European heartjournal 24(13):1231-1243.
6. Schlotter F, et al. (2018) Spatiotemporal Multi-omics Mapping Generates a Molecular Atlas of the Aortic Valve and Reveals Networks Driving Disease. Circulation.
7. Aikawa E & Libby P (2017) A Rock and a Hard Place: Chiseling Away at the Multiple Mechanisms of Aortic Stenosis. Circulation 135(20):1951-1955.
8. Hinton R B, et al. (2006) Extracellular Matrix Remodeling and Organization in Developing and Diseased Aortic Valves. Circulation Research 98(11):1431-1438.
9. Walker G A, Masters K S, Shah D N, Anseth K S, & Leinwand L A (2004) Valvular Myofibroblast Activation by Transforming Growth Factor-β. Circulation Research 95(3):253.
10. Towler D A (2013) Molecular and cellular aspects of calcific aortic valve disease. Circulation research 113(2):198-208.
11. Blevins T L, et al. (2008) Mitral valvular interstitial cells demonstrate regional, adhesional, and synthetic heterogeneity. Cells, tissues, organs 187(2):113-122.
12. Nomura A, et al. (2013) CD34-negative mesenchymal stem-like cells may act as the cellular origin of human aortic valve calcification. Biochemical and Biophysical Research Communications 440(4):780-785.
13. Chen J-H, Yip C Y Y, Sone E D, & Simmons C A (2009) Identification and characterization of aortic valve mesenchymal progenitor cells with robust osteogenic calcification potential. The American journal ofpathology 174(3):1109-1119.
14. Wang H, Sridhar B, Leinwand L A, & Anseth K S (2013) Characterization of cell subpopulations expressing progenitor cell markers in porcine cardiac valves. PloS one 8(7):e69667-e69667.
15. Gostjeva E V, et al. (2009) Metakaryotic stem cell lineages in organogenesis of humans and other metazoans. Organogenesis 5(4):191-200.
16. Icer M A & Gezmen-Karadag M (2018) The multiple functions and mechanisms of osteopontin. Clinical Biochemistry 59:17-24.
17. Freise C, Bobb V, & Querfeld U (2017) Collagen XIV and a related recombinant fragment protect human vascular smooth muscle cells from calcium-/phosphate-induced osteochondrocytic transdifferentiation. Experimental Cell Research 358(2):242-252.
18. Jordan A R, Racine R R, Hennig M J P, & Lokeshwar V B (2015) The Role of CD44 in Disease Pathophysiology and Targeted Treatment. Frontiers in immunology 6:182-182.
19. Cho H-J, Lee J-W, Cho H-J, Lee C-S, & Kim H-S (2018) Identification of Adult Mesodermal Progenitor Cells and Hierarchy in Atherosclerotic Vascular Calcification. STEM CELLS 36(7):1075-1096.
20. Moravcikova E, Meyer E M, Corselli M, Donnenberg V S, & Donnenberg A D (2018) Proteomic Profiling of Native Unpassaged and Culture-Expanded Mesenchymal Stromal Cells (MSC). Cytometry PartA 93(9):894-904.
21. Kim J & Hematti P (2009) Mesenchymal stem cell-educated macrophages: A novel type of alternatively activated macrophages. Experimental Hematology 37(12):1445-1453.
22. Mahmut A, Boulanger M-C, Bouchareb R, Hadji F, & Mathieu P (2015) Adenosine derived from ecto-nucleotidases in calcific aortic valve disease promotes mineralization through A2a adenosine receptor. Cardiovascular Research 106(1):109-120.
23. Kaniewska-Bednarczuk E, et al. (2018) CD39 and CD73 in the aortic valve biochemical and immunohistochemical analysis in valve cell populations and its changes in valve mineralization. Cardiovascular Pathology 36:53-63.
24. Zukowska P, et al. (2017) Deletion of CD73 in mice leads to aortic valve dysfunction. Biochimica et Biophysica Acta (BBA)—Molecular Basis of Disease 1863(6):1464-1472.
25. Baird A, et al. (2018) Osteoblast differentiation of equine induced pluripotent stem cells. Biology Open 7(5).
26. Wang W, et al. (2014) Mesenchymal Stem Cells Recruited by Active TGFβ Contribute to Osteogenic Vascular Calcification. Stem Cells and Development 23(12):1392-1404.
27. Oh J-E, et al. (2011) PlexinA2 mediates osteoblast differentiation via regulation of Runx2. Journal of Bone and Mineral Research 27(3):552-562.
28. Iwata J-i, et al. (2010) Transforming Growth Factor-O Regulates Basal Transcriptional Regulatory Machinery to Control Cell Proliferation and Differentiation in Cranial Neural Crest-derived Osteoprogenitor Cells. Journal of Biological Chemistry 285(7):4975-4982.
29. Pena-Silva R A, Miller J D, Chu Y, & Heistad D D (2009) Serotonin produces monoamine oxidase-dependent oxidative stress in human heart valves. American journal of physiology. Heart and circulatory physiology 297(4):H1354-1360.
30. Liu M, Luo M, Sun H, Ni B, & Shao Y (2017) Integrated Bioinformatics Analysis Predicts the Key Genes Involved in Aortic Valve Calcification: From Hemodynamic Changes to Extracellular Remodeling. The Tohoku journal of experimental medicine 243(4):263-273.
31. Yamamoto S, et al. (2008) Cthrc1 Selectively Activates the Planar Cell Polarity Pathway of Wnt Signaling by Stabilizing the Wnt-Receptor Complex. Developmental Cell 15(1):23-36.
32. Albanese I, et al. (2017) Role of Noncanonical Wnt Signaling Pathway in Human Aortic Valve Calcification. Arteriosclerosis, thrombosis, and vascular biology 37(3):543-552.
33. Pyagay P, et al. (2005) Collagen triple helix repeat containing 1, a novel secreted protein in injured and diseased arteries, inhibits collagen expression and promotes cell migration. Circ Res 96(2):261-268.
34. Lindman B R, et al. (2016) Calcific aortic stenosis. Nat Rev Dis Primers 2:16006-16006.
35. Walmsley G G, et al. (2015) High-Throughput Screening of Surface Marker Expression on Undifferentiated and Differentiated Human Adipose-Derived Stromal Cells. Tissue engineering. PartA 21(15-16):2281-2291.
36. Baer P C, et al. (2012) Comprehensive Phenotypic Characterization of Human Adipose-Derived Stromal/Stem Cells and Their Subsets by a High Throughput Technology. Stem Cells and Development 22(2):330-339.
37. Dimitrios S. Monos R J W (2018) Clinical Immunology—Principles and Practice. ScienceDirect (Fifth Edition):79-92.e71.
38. Millar S A, Patel H, Anderson S I, England T J, & O'Sullivan S E (2017) Osteocalcin, Vascular Calcification, and Atherosclerosis: A Systematic Review and Meta-analysis. Frontiers in Endocrinology 8:183.
39. Chellan B, Rojas E, Zhang C, & Hofmann Bowman M A (2018) Enzyme-modified non-oxidized LDL (ELDL) induces human coronary artery smooth muscle cell transformation to a migratory and osteoblast-like phenotype. Scientific Reports 8(1):11954.
40. Zhiduleva E V, et al. (2018) Cellular Mechanisms of Aortic Valve Calcification. Bulletin of Experimental Biology and Medicine 164(3):371-375.
41. Boyer L A, et al. (2005) Core transcriptional regulatory circuitry in human embryonic stem cells. Cell 122(6):947-956.
42. Li S-J, et al. (2017) Activated p300 acetyltransferase activity modulates aortic valvular calcification with osteogenic transdifferentiation and downregulation of Klotho. International Journal of Cardiology 232:271-279.
43. Yu P, Nguyen B T, Tao M, Campagna C, & Ozaki C K (2010) Rationale and practical techniques for mouse models of early vein graft adaptations. Journal of vascular surgery 52(2):444-452.
44. Nishimura I, et al. (2015) Effect of osteogenic differentiation medium on proliferation and differentiation of human mesenchymal stem cells in three-dimensional culture with radial flow bioreactor. Regenerative Therapy 2:24-31.
45. Sage A P, Lu J, Tintut Y, & Demer L L (2011) Hyperphosphatemia-induced nanocrystals upregulate the expression of bone morphogenetic protein-2 and osteopontin genes in mouse smooth muscle cells in vitro. Kidney international 79(4):414-422.
46. Ciceri P, et al. (2016) Osteonectin (SPARC) Expression in Vascular Calcification: In Vitro and Ex Vivo Studies. Calcified Tissue International 99(5):472-480.
47. Bendall S C, Nolan G P, Roederer M, & Chattopadhyay P K (2012) A deep profiler's guide to cytometry. Trends in Immunology 33(7):323-332.
48. Qiu P (2017) Toward deterministic and semi-automated SPADE analysis( ). Cytometry. Part A: the journal of the International Society for Analytical Cytology 91(3):281-289.
49. Zilionis R, et al. (2017) Single-cell barcoding and sequencing using droplet microfluidics. Nat Protoc 12(1):44-73.
50. Menche J, et al. (2015) Uncovering disease-disease relationships through the incomplete interactome. Science 347(6224):1257601.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for the treatment of a disorder associated with aortic valve calcification, the method comprising administering a therapeutically effective amount of an inhibitor of monoamine oxidase subtype A (MAOA), to a subject who is in need of, or who has been determined to be in need of, such treatment.

2. The method of claim 1, further comprising identifying the subject as having a disorder associated with aortic valve calcification.

3. The method of claim 1, wherein the subject has calcific aortic stenosis (CAS), coronary artery disease, carotid artery disease, peripheral artery disease, vein graft failure, arteriovenous fistula, and scleroderma.

4. The method of claim 3, wherein the subject is a mammal, preferably a human.

5. The method of claim 1, wherein the inhibitor of MAOA is a small molecule inhibitor.

6. The method of claim 5, wherein the small molecule inhibitor of MAOA is selected from the group consisting of Befloxatone (MD370503); Bifemelane (Alnert, Celeport); Brofaromine (Consonar); Cimoxatone (MD 780515); Clorgyline (or Clorgiline); Methylene Blue; Minaprine (Cantor); Moclobemide (Aurorix, Manerix); Phenelzine (Nardil); Pirlindole (Pirazidol); Toloxatone (Humoryl); Tyrima (CX 157); Tranylcypromine (nonselective and irreversible, Parnate); Isocarboxazid (1-benzyl-2-(5-methyl-3-isoxazolylcarbonyl)hydrazine-isocarboxazid, Marplan, Marplon, Enerzer); Molindone; Ladostigil; VAR 10303; M30; Hydralazine; Phenelzine; quinacrine; and salts and combinations thereof.

7. The method of claim 1, wherein the inhibitor of MAOA is an inhibitory nucleic acid targeting MAOA.

8. The method of claim 7, wherein the inhibitory nucleic acid targeting MAOA is an antisense RNA; antisense DNA; chimeric antisense oligonucleotide; antisense oligonucleotide comprising modified linkages; interference RNA (RNAi); short interfering RNA (siRNA); a short, hairpin RNA (shRNA); small RNA-induced gene activation (RNAa); small activating RNAs (saRNAs); gapmer; mixmer; locked nucleic acid (LNA); or peptide nucleic acid (PNA).

9. The method of claim 7, wherein the inhibitory nucleic acid is modified.

10. The method of claim 9, wherein the inhibitory nucleic acid comprises a modified backbone, preferably comprising phosphorothioates, phosphotriesters, methyl phosphonates, short chain alkyl or cycloalkyl intersugar linkages or short chain heteroatomic or heterocyclic intersugar linkages.

11. The method of claim 9, wherein the inhibitory nucleic acid comprises one or more modified nucleotides, optionally comprising one or more modified nucleobases nucleotides modified at the 2′ position of the sugar.

12.-22. (canceled)
